(19) United States
(12) Patent Application Publication
(10) Pub. No.: US 2024/0285638 A1
(43) Pub. Date: Aug. 29, 2024

(54) METHODS FOR TREATING FACIOSCAPULOHUMERAL MUSCULAR DYSTROPHY (FSHD)

(71) Applicant: RESEARCH INSTITUTE AT NATIONWIDE CHILDREN'S HOSPITAL, Columbus, OH (US)

(72) Inventors: Scott Quenton Harper, Powell, OH (US); Jocelyn Eidahl, Delaware, OH (US); Lindsay Wallace, Columbus, OH (US); Renatta Knox, Columbus, OH (US)

(21) Appl. No.: 18/573,684

(22) PCT Filed: Jun. 24, 2022
(86) PCT No.: PCT/US2022/034872

§ 371 Date: Dec. 22, 2023

Related U.S. Application Data
(60) Provisional application No. 63/214,587, filed on Jun. 24, 2021.

(51) Int. Cl. A61K 31/52; A61K 31/192; A61P 21/00

(52) U.S. Cl. CPC A61K 31/52; A61K 31/192; A61P 21/00

(57) ABSTRACT

Disclosed herein are methods and uses for treating, ameliorating, delaying the progression of, and/or preventing a muscular dystrophy or a cancer including, but not limited to, facioscapulohumeral muscular dystrophy (FSHD) or a sarcoma. More particularly, disclosed herein are methods of using small molecule protein arginine methylation (PRMT) inhibitors, and uses of these inhibitors, for inhibiting methylation of amino acids, e.g., arginine, in the double homeobox 4 (DUX4) protein. Even more particularly, the disclosure provides methods of using such methylation inhibitors or arginine methylation inhibitors for inhibiting methylation of the DUX4 protein resulting in reduced DUX4-activated cell death, including reduced DUX4-activated muscle cell death and/or reduced DUX4 target gene activation. The disclosure provides, in some aspects, methods of using protein methylation inhibitors including, but not limited to salvianolic acid A (SAA), or a derivative thereof, or adenosine dialdehyde (ADOX), or a derivative thereof for inhibiting methylation of arginine residues of the DUX4 protein in cells in vitro, ex vivo, or in vivo in the cells of a subject at risk of or suffering from a muscular dystrophy or a cancer associated with DUX4 overexpression.

### INCORPORATION BY REFERENCE OF THE SEQUENCE LISTING

This application contains, as a separate part of disclosure, a Sequence Listing in computer-readable form (Filename: 56936_Seqlisting.txt; Size: 6,005 bytes; Created: Jun. 22, 2022) which is incorporated by reference herein in its entirety.

### FIELD

This disclosure relates to the field of the treatment of a muscular dystrophy or a cancer including, but not limited to, facioscapulohumeral muscular dystrophy (FSHD) or a sarcoma. More particularly, the disclosure provides methods for treating, ameliorating, delaying the progression of, and/or preventing a muscular dystrophy or a cancer including, but not limited to, FSHD or a sarcoma. Specifically, the disclosure provides methods for inhibiting the methylation of arginine residues of the double homeobox 4 (DUX4) protein by administering a small molecule inhibitor of protein arginine methyl transferase (PRMT). More specifically, the disclosure provides PRMT inhibitors for decreasing DUX4-induced cell death and/or DUX4-induced gene activation and methods of using said PRMT inhibitors to treat a subject suffering from or at risk of suffering from a muscular dystrophy or a cancer associated with DUX4 overexpression.

### BACKGROUND

Muscular dystrophies (MDs) are a group of genetic diseases. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

Facioscapulohumeral muscular dystrophy (FSHD) is one of the most common forms of muscular dystrophy, affecting an estimated 870,000 people worldwide. It is a significant cause of morbidity with many patients experiencing debilitating pain, fatigue, and progressive asymmetric muscle weakness¹. There are currently no disease modifying treatments and therapy development remains a critical unmet need. FSHD is caused by de-repression of the transcription factor DUX4, which is normally expressed during early embryonic development and in the testes, and is epigenetically silenced in most somatic tissues^{2,3}. Aberrant expression of DUX4 is toxic to muscle in vitro and in vivo⁴. DUX4 activates numerous pathways that may contribute to myofiber toxicity, [symbol] stress, immune activation, and apoptosis⁵. While significant progress has been made in linking DUX4 function to toxicity, the contribution of DUX4 regulation to FSHD pathology is unknown. Despite progress in the FSHD field, there are still no approved treatments for FSHD, and therapeutic development remains a critical need in the field. There remains a need in the art for methods for treating muscular dystrophies including, but not limited to, FSHD.

Post-translational modifications (PTMs) play a critical role in protein regulation and function. Arginine methylation is a PTM that has been implicated in regulating proteins involved in transcription, pre-mRNA splicing as well as myogenesis and skeletal muscle regeneration^{6,7}. Small molecule inhibitors of protein arginine methyltransferases (PRMTs) are being explored in cancer^{6,8}, however it is unknown whether arginine methylation inhibitors could be protective in FSHD disease models. This disclosure provides methods for DUX4 regulation at the level of post-translational modification and for the therapeutic application of arginine methylation inhibition in FSHD disease. The disclosure provides methods of using inhibitors of PRMTs to inhibit DUX4-induced cell death and DUX4-induced gene target activation in muscle cells and in subjects with MD, and FSHD.

### SUMMARY

The disclosure provides methods and uses for inhibiting DUX4 target gene expression (or target gene transactivation) and decreased DUX4-induced apoptotic cell death for treating, ameliorating, delaying the progression of, and/or preventing a muscular dystrophy (MD). In some aspects, the muscular dystrophy is facioscapulohumeral dystrophy (FSHD). More specifically, the disclosure provides small molecule inhibitors of protein arginine methyltransferases (PRMTs or RMTs) to inhibit the methylation of arginine residues of the double homeobox 4 (DUX4) protein in a cell or in the cell of a subject suffering from a muscular dystrophy associated with DUX4 overexpression. The disclosure provides such arginine methylation inhibitors as a means for regulating DUX4-mediated toxicity and as a means for the treatment of muscular dystrophy including, but not limited to, FSHD.

The disclosure provides a method of inhibiting arginine methylation of a double homeobox 4 (DUX4) protein in a cell comprising contacting the cell with an effective amount of at least one inhibitor of protein arginine methyl transferase (PRMT), wherein the inhibitor of PRMT is at least one of Compound I and Compound II, or a salt, hydrate, or stereoisomer thereof:

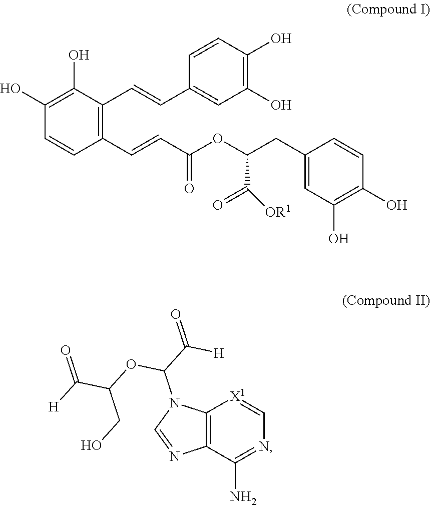

wherein R¹ is H or C₁-C₆ alkyl; and X¹ is CH or N.

The disclosure provides a method of decreasing double homeobox 4 (DUX4)-associated apoptotic cell death and/or decreasing DUX4 target gene activation in a cell comprising contacting the cell with an effective amount of at least one of Compound I and Compound II, or a salt, hydrate, or stereoisomer thereof:

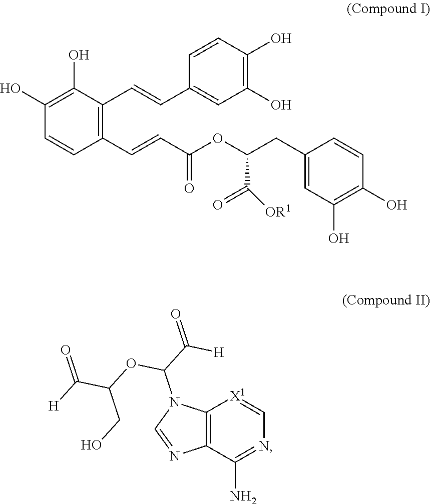

wherein R¹ is H or C₁-C₆ alkyl; and X¹ is CH or N.

The disclosure provides a method of treating an arginine methyl transferase (RMT) disorder in a patient in need thereof comprising administering to the patient a therapeutically effective amount of at least one of Compound I and Compound II, or a salt, hydrate or stereoisomer thereof:

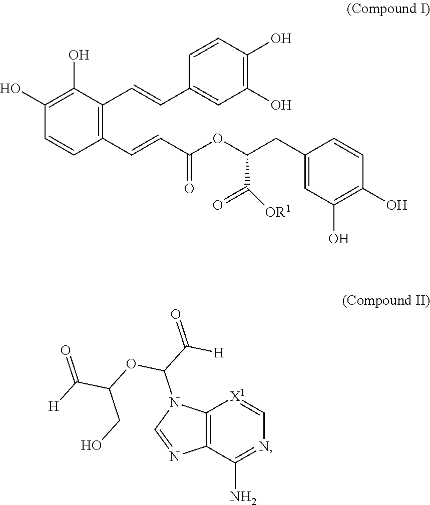

wherein R¹ is H or C₁-C₆ alkyl; and X¹ is CH or N.

In some aspects, the cell is in a subject at risk of or suffering from a muscular dystrophy. In some aspects, the subject is a human subject.

In some aspects, the PRMT disorder is a muscular dystrophy. In some aspects, the muscular dystrophy is a facioscapulohumeral muscular dystrophy (FSHD).

In some aspects, Compound I is Compound Ia:

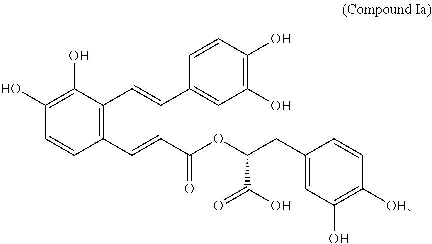 [SMILES: O=C(/C=C/c1ccc(O)c(O)c1/C=C/c1ccc(O)c(O)c1)O[C@H](Cc1ccc(O)c(O)c1)C(=O)O]

or a salt, a hydrate, or a stereoisomer thereof.

In some aspects, Compound Ia is a hydrate of formula:

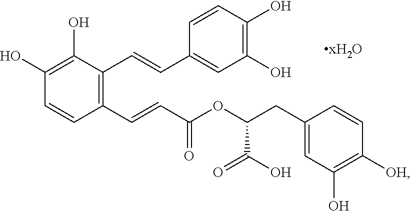 [SMILES: O=C(/C=C/c1ccc(O)c(O)c1/C=C/c1ccc(O)c(O)c1)O[C@H](Cc1ccc(O)c(O)c1)C(=O)O]

and x is 0.5 to 10.

In some aspects, Compound II is Compound IIa:

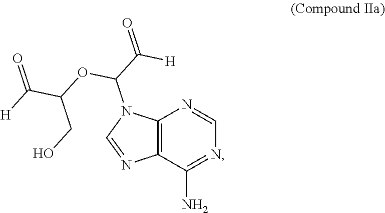 [SMILES: Nc1ncnc2c1ncn2C(C=O)OC(C=O)CO]

or a salt, a hydrate, or a stereoisomer thereof.

The disclosure provides a use of at least one of Compound I and Compound II, or a salt, hydrate or stereoisomer thereof:

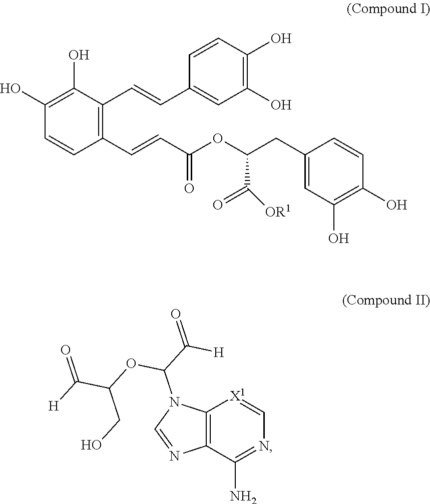

wherein R¹ is H or C₁-C₆ alkyl; and X¹ is CH or N for the preparation of a medicament for inhibiting arginine methylation of a double homeobox 4 (DUX4) protein in a cell.

The disclosure provides a use of at least one of Compound I and Compound II, or a salt, hydrate or stereoisomer thereof:

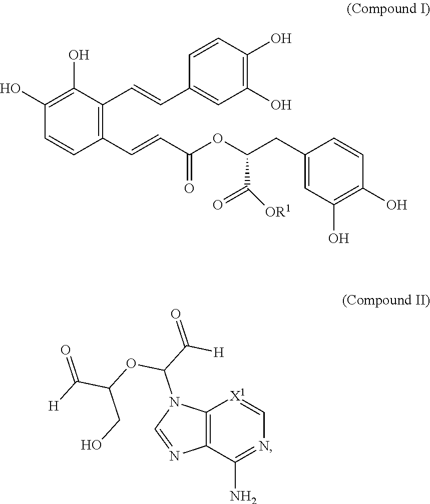

wherein R¹ is H or C₁-C₆ alkyl; and X¹ is CH or N for the preparation of a medicament for decreasing double homeobox 4 (DUX4)-associated apoptotic cell death and/or decreasing DUX4 target gene activation in a cell.

The disclosure provides a use of at least one of Compound I and Compound II, or a salt, hydrate or stereoisomer thereof:

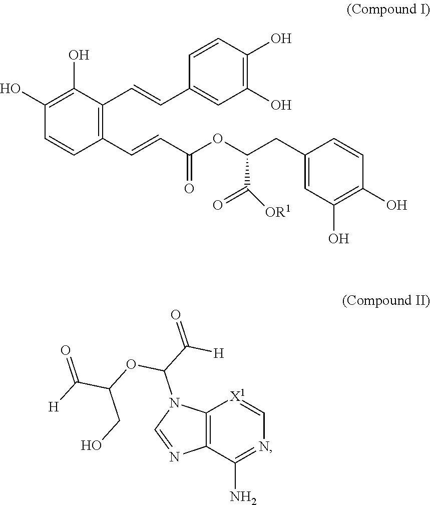

wherein R¹ is H or C₁-C₆ alkyl; and X¹ is CH or N for the preparation of a medicament for the preparation of a medicament for treating an arginine methyl transferase (RMT) disorder in a patient in need thereof.

In some aspects, the cell is in a subject at risk of or suffering from a muscular dystrophy. In some aspects, the subject is a human subject.

In some aspects, the PRMT disorder is a muscular dystrophy. In some aspects, the muscular dystrophy is a facioscapulohumeral muscular dystrophy (FSHD).

In some aspects, the decrease in DUX4 target gene activation is demonstrated by the decreased expression of DUX4 target genes including, but not limited to, PRAME Family Member 12 (PRAMEF12), Zinc finger and SCAN domain-containing protein 4 (ZSCAN4), Tripartite Motif Containing 43 (TRIM43), and Leucine Twenty Homeobox (LEUTX).

In some aspects, Compound I is Compound Ia:

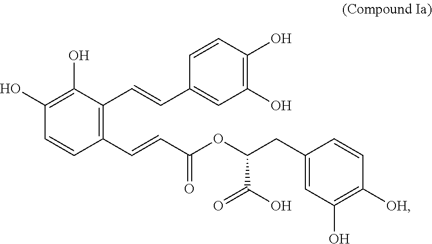 [SMILES: O=C(/C=C/c1ccc(O)c(O)c1/C=C/c1ccc(O)c(O)c1)O[C@H](Cc1ccc(O)c(O)c1)C(=O)O]

or a salt, a hydrate, or a stereoisomer thereof.

In some aspects, Compound Ia is a hydrate of formula:

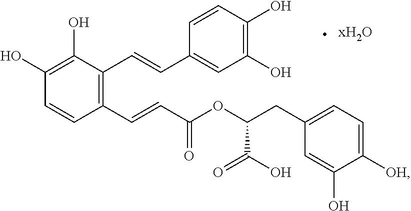 [SMILES: O=C(/C=C/c1ccc(O)c(O)c1/C=C/c1ccc(O)c(O)c1)O[C@H](Cc1ccc(O)c(O)c1)C(=O)O]

and x is 0.5 to 10.

In some aspects Compound II is Compound IIa:

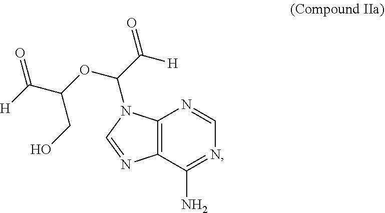 [SMILES: Nc1ncnc2c1ncn2C(C=O)OC(C=O)CO]

or a salt, a hydrate, or a stereoisomer thereof.

The disclosure provides methods and uses, as described herein, wherein the small molecule inhibitor of the PRMT is Compound A or a salt, a hydrate, or a stereoisomer thereof and/or Compound B, or a salt, a hydrate, or a stereoisomer thereof:

The disclosure also provides methods and uses, wherein the small molecule inhibitor of the PRMT, or a composition comprising the small molecule inhibitor of the PRMT, or medicament comprising the small molecule inhibitor of the PRMT is formulated for intramuscular injection, transdermal transport or injection into the blood stream.

Further aspects and advantages of the disclosure will be apparent to those of ordinary skill in the art from a review of the following detailed description, taken in conjunction with the drawings. It should be understood, however, that the detailed description (including the drawings and the specific examples), while indicating embodiments of the disclosed subject matter, are given by way of illustration only, because various changes and modifications within the spirit and scope of the disclosure will become apparent to those skilled in the art from this detailed description.

### BRIEF DESCRIPTION OF THE DRAWINGS

FIGS. 1A-B show DUX4 Arginine methylation sites (FIG. 1A) and a mutagenesis strategy (FIG. 11B). FIG. 1A provides a schematic of DUX4 arginine methylation PTM sites identified by mass spectrometry. FIG. 1B provides a mutagenesis overview.

FIG. 2 shows that the DUX4 PTM R71A mutant protects against [symbol] HEK293 cells were transfected with wild type or DUX4 R71A mutant and the caspase assay was performed 48 hours later to assess for cell death.

FIGS. 3A-B show decreased DUX4 target gene expression in the R71A PTM mutant. FIG. 3A shows HEK293 cells and FIG. 3B shows myoblasts transfected with wild type DUX4, empty vector (pCIneo) or DUX4 R71A. Quantitative RT-PCR was performed 24 hours later for DUX4 target genes PRAMF12, ZSCAN4, TRIM43 and LEUTX, indicating that the R71A PTM mutant had decreased levels of DUX4 target gene expression.

FIGS. 4A-B show that the DUX4 R71A mutant has decreased transactivation. FIG. 4A shows the localization of R71 in the crystal structure of DUX4 with DNA. FIG. 4B shows GFP expression visualized after 24 hours after HEK293 cells co-transfected with a DUX4-activated fluorescence reporter (DRE) and wild type DUX4, DUX4 R71A or empty vector. The DUX4 R71A mutant demonstrated decreased transactivation compared to the controls.

FIGS. 5A-B show PRMT1 is a DUX4 complex member. FIG. 5A provides a schematic of the RIME assay. FIG. 5B shows co-immunoprecipitation of DUX4 and PRMT1 in HEK293 cells.

FIGS. 6A-B show that arginine methylation inhibitors protect against cell death in myoblasts. Myoblasts were transfected with DUX4 or no DNA in the presence or absence of increasing concentrations of SAA (FIG. 6A) or AdOX (FIG. 6B) and the caspase assay was performed 48 hours later.

### DETAILED DESCRIPTION

The disclosure provides methods and uses for treating, ameliorating, delaying the progression of, and/or preventing a muscular dystrophy or a cancer including, but not limited to, facioscapulohumeral muscular dystrophy (FSHD) or a sarcoma. More particularly, disclosed herein are methods and uses of protein arginine methylation inhibitors for inhibiting methylation of arginine in the double homeobox 4 (DUX4) protein. Even more particularly, the disclosure provides methods of using protein arginine methylation inhibitors for inhibiting methylation of the DUX4 protein resulting in reduced DUX4-activated cell death, including reduced DUX4-activated muscle cell death.

The disclosure therefore provides methods of using protein arginine methylation inhibitors including, but not limited to salvianolic acid A (SAA), or a derivative thereof, or adenosine dialdehyde (ADOX), or a derivative thereof for inhibiting the methylation of arginine residues in the DUX4 protein in cells and, in some instances, in cells of a subject at risk of or suffering from a muscular dystrophy. In some aspects, such muscular dystrophy is associated with DUX4 protein overexpression. In some aspects, such [symbol] FSHD.

The disclosure provides methods for repressing or inhibiting methylation of arginine amino acids in the DUX4 protein because such inhibition of DUX4 methylation in muscle cells is associated with decreased DUX4-induced cell death. Thus, in some aspects, the products and methods described herein are used in treating, ameliorating, delaying the progression of, and/or preventing muscular dystrophies associated with elevated levels of DUX4 protein including, but not limited to, FSHD.

The DUX4 gene encodes an approximately 45 kDA protein; see UniProtKB-Q9UBX2 (DUX4_HUMAN). De-repression of the DUX4 gene is involved in disease pathogenesis of FSHD. De-repression can occur through two known mechanisms: D4Z4 repeat contraction, or mutation in chromatin modifier genes SMCHD1 or DNMT3B. For the former, in unaffected subjects, the D4Z4 array consists of 11-100 repeats, while in FSHD1 patients, the array is reduced to 1-10 repeats (PubMed:19320656). Either condition can cause DNA hypomethylation at chromosome 4q35, thereby creating a chromosomal environment permissive for DUX4 expression.

DUX4 is located in D4Z4 macrosatellite repeats, which are epigenetically repressed in somatic tissues. D4Z4 chromatin relaxation in FSHD1 results in inefficient epigenetic repression of DUX4 and a variegated pattern of DUX4 protein expression in a subset of skeletal muscle nuclei. Ectopic expression of DUX4 in skeletal muscle activates the expression of stem cell and germline genes, and, when overexpressed in somatic cells, DUX4 can ultimately lead to cell death.

Each D4Z4 repeat unit has an open reading frame (named DUX4) that encodes two homeoboxes; the repeat-array and ORF is conserved in other mammals. The encoded protein has been reported to function as a transcriptional activator of numerous genes, including some considered to be FSHD disease biomarkers, including ZSCAN4, PRAMEF12, TRIM43, and MBD3L2 (PMID: 24861551). Contraction of the macrosatellite repeat causes autosomal dominant FSHD. Alternative splicing results in multiple transcript variants.

In some embodiments of the disclosure, the DUX4 nucleic acid and protein are provided. In some aspects, the nucleic acid encoding human DUX4 is set forth in the nucleotide sequence set forth in SEQ ID NO: 1. In some aspects, the amino acid sequence of human DUX4 is set forth in the amino acid sequence set forth in SEQ ID NO: 2. In various aspects, the methods of the disclosure also target isoforms and variants of the DUX4 polypeptide comprising the amino acid sequence set forth in SEQ ID NO: 2. In some aspects, the methods of the disclosure target isoforms and variants of [symbol] forth in the amino acid sequence set forth in SEQ ID NO: 2. In some aspects, the variants comprise 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the amino acid sequence set forth in SEQ ID NO: 2.

| SEQ ID NO: | Sequence |
|---|---|
| 1 | DUX4 NT: |
| | atggccctcccgacaccctcggacagcaccctccccgcggaagcccggggacgaggacggcgacgga |
| | gactcgtttggaccccgagccaaagcgaggccctgcgagcctgctttgagcggaacccgtacccgggcat |
| | cgccaccagagaacggctggcccaggccatcggcattccggagcccagggtccagatttggtttcagaat |
| | gagaggtcacgccagctgaggcagcaccggcgggaatctcggccctggcccgggagacgcggcccgc |
| | cagaaggccggcgaaagcggaccgccgtcaccggatcccagaccgccctgctcctccgagcctttgaga |
| | aggatcgctttccaggcatcgccgcccgggaggagctggccagagagacgggcctcccggagtccagg |
| | attcagatctggtttcagaatcgaagggccaggcacccgggacagggggcagggcgcccgcgcaggc |
| | aggcggcctgtgcagcgcggcccccggcgggggtcaccctgctccctcgtgggtcgccttcgcccacacc |
| | ggcgcgtggggaacggggcttcccgcaccccacgtgccctgcgcgcctggggctctcccacagggggctt |
| | tcgtgagccaggcagcgagggccgcccccgcgctgcagcccagccaggccgcgccggcagaggggat |
| | ctcccaacctgccccggcgcgcggggatttcgcctacgccgccccggctcctccggacggggcgctctcc |
| | caccctcaggctcctcggtggcctccgcacccgggcaaaagccgggaggaccgggacccgcagcgcg |
| | acggcctgccgggcccctgcgcggtggcacagcctgggcccgctcaagcggggccgcagggccaagg |
| | ggtgcttgcgccacccacgtcccaggggagtccgtggtggggctggggccggggtccccaggtcgccgg |
| | ggcggcgtgggaaccccaagccggggcagctccacctccccagcccgcgcccccggacgcctccgcct |
| | ccgcgcggcaggggcagatgcaaggcatcccggcgccctcccaggcgctccaggagccggcgccctg |
| | gtctgcactcccctgcggcctgctgctggatgagctcctggcgagcccggagtttctgcagcaggcgcaacc |
| | tctcctagaaacggaggccccgggggagctggaggcctcggaagaggccgcctcgctggaagcacccc |
| | tcagcgaggaagaataccgggctctgctggaggagctttag |
| 2 | DUX4 AA: |
| | MALPTPSDSTLPAEARGRGRRRRLVWTPSQSEALRACFERNPYPGIATRER |
| | LAQAIGIPEPRVQIWFQNERSRQLRQHRRESRPWPGRRGPPEGRRKRTAV |
| | TGSQTALLLRAFEKDRFPGIAAREELARETGLPESRIQIWFQNRRARHPGQG |
| | GRAPAQAGGLCSAAPGGGHPAPSWVAFAHTGAWGTGLPAPHVPCAPGAL |
| | PQGAFVSQAARAAPALQPSQAAPAEGISQPAPARGDFAYAAPAPPDGALSH |
| | PQAPRWPPHPGKSREDRDPQRDGLPGPCAVAQPGPAQAGPQGQGVLAPP |
| | TSQGSPWWGWGRGPQVAGAAWEPQAGAAPPPQPAPPDASASARQGQM |
| | QGIPAPSQALQEPAPWSALPCGLLLDELLASPEFLQQAQPLLETEAPGELEA |
| | SEEAASLEAPLSEEEYRALLEEL |

There is currently no treatment for FSHD, and despite its relative abundance among the muscular dystrophies, very few FSHD-targeted translational studies have been published. Several FSHD candidate genes have been identified, but numerous recent studies support that the primary contributor to FSHD pathogenesis is the pro-apoptotic DUX4 gene, which encodes a transcription factor. Thus, in the simplest terms, DUX4-overexpression is a primary pathogenic insult underlying FSHD (Chen et al., (2016) Mol Ther 24, 1405-1411; Ansseau et al. (2017) Genes (Easel) 8; Lek et al. (2020) Sci Transl Med 12; Himeda et al. (2016) Mol Ther 24, 527-535; DeSimone et al. (2019) Sci Adv 5, 12; Lim et al. (2020) Proc Natl Acad Sci USA 117, 16509-16515; Wallace et al. (2018), [symbol] al. (2020) J Pharmacol Exp Ther. September; 374(3):489-498).

In some embodiments, the disclosure provides small molecule inhibitors of arginine methylation for use in inhibiting or downregulating DUX4-induced cell death and/or for treating a muscular dystrophy associated with DUX4-induced cell death, such as FSHD. Arginine methylation is enzymatically catalyzed by the family of protein arginine methyltransferases (PRMTs) which can either activate or repress gene expression depending on cellular contexts. Given the strong correlation of PRMTs with pathophysiology, great interest is seen in understanding molecular mechanisms of PRMTs in diseases and in developing potent PRMT inhibitors.

The disclosure provides small molecule inhibitors of arginine methylation (PRMT inhibitors or RMT inhibitors) in order to inhibit arginine methylation of DUX4 in order to decrease DUX4-mediated cell death and DUX4-mediated gene activation associated with a muscular dystrophy including, but not limited to FSHD. As used herein, the terms “protein arginine methyltransferase (PRMT) inhibitor” or “inhibitor of PRMT” and the terms “arginine methyltransferase (RMT) inhibitor” or “inhibitor of RMT” are used interchangeably. In some aspects, such small molecule PRMT inhibitor is a salvianolic acid, or a derivative thereof, or an adenosine dialdehyde (ADOX), or a derivative thereof.

In some aspects, the small molecule inhibitor of arginine methyl transferase (PRMT) is at least one of Compound I and Compound II, or a salt, hydrate, or stereoisomer thereof:

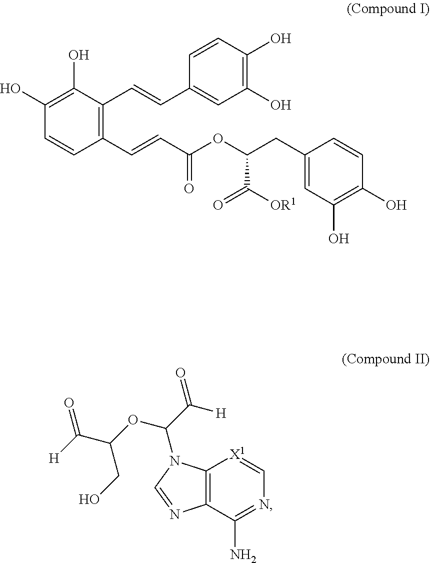

wherein R¹ is H or C₁-C₆ alkyl; and X¹ is CH or N.

Suitable C₁-C₆ alkyl groups include linear and branched C₁-C₆ alkyl groups, including but not limited to methyl, ethyl, n-propyl, n-butyl, n-pentyl, hexyl, isopropyl, isobutyl, isopentyl, isohexyl, sec-butyl, sec-pentyl, 3-pentyl, sec-isopentyl, sec-hexyl, neo-pentyl, tert-butyl, tert-pentyl, and tert-hexyl.

In some aspects, Compound I is Compound Ia:

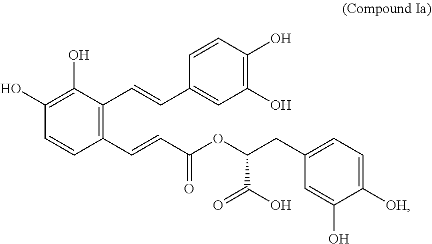 [SMILES: O=C(/C=C/c1ccc(O)c(O)c1/C=C/c1ccc(O)c(O)c1)O[C@H](Cc1ccc(O)c(O)c1)C(=O)O]

or a salt, a hydrate, or a stereoisomer thereof.

In some aspects, Compound Ia is a hydrate of formula:

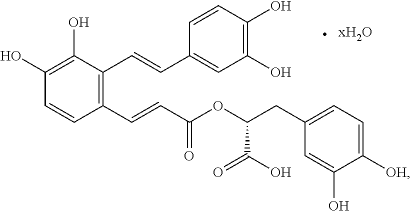 [SMILES: O=C(/C=C/c1ccc(O)c(O)c1/C=C/c1ccc(O)c(O)c1)O[C@H](Cc1ccc(O)c(O)c1)C(=O)O]

and x is 0.5 to 10.

In some aspects, Compound I or Ia is a salvianolic acid or a derivative thereof.

In some aspects, Compound II is Compound IIa:

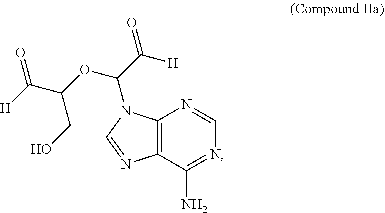 [SMILES: Nc1ncnc2c1ncn2C(C=O)OC(C=O)CO]

or a salt, a hydrate, or a stereoisomer thereof.

In some aspects, Compound II or IIa is an adenosine dialdehyde (ADOX) or a derivative thereof.

Salvia miltiorrhiza (SM), has long been used in traditional Chinese medicine for treatment of cardiovascular and hepatic diseases. Extracts from the plant confer potent hepatoprotective activity both in vitro and in vivo. Hase et al., Planta Med. 63: 22-6 (1997). Magnesium lithospermate B may be one of the main active components of SM that protects the liver (Liu et al., Chung Kuo Chung Hsi I Chih Ho Tsa Chih 13: 352-3, 326 (1993)). SM also contains antioxidants that apparently aid membrane damage repair when treating viral myocarditis (Meng et al., Chung Kuo Chung Hsi I Chieh Ho Tsa Chih 12: 345-7, 324-5 (1992)). Patients suffering from chronic hepatitis B have responded to treatment with SM and/or Polyporus Umbellatus polysaccharidc (PUP) (Xiong, Chung Kuo Chung Hsi I Chieh Ho Tsa Chih 13: 33-5, 516-7 (1993)). Herbal extracts from SM also have [symbol] HIV activity (U.S. Pat. No. 5,178,865) and anti-hepatitis activity (International PCT Application 98/24460; Chinese Patent Application Nos. 1,192,922 and 1,192,918). Antiviral agents active against herpes, polio, measles, varicella zoster, cytomegalovirus, DNA viruses and RNA viruses have been described which contain at least one crude drug from the root of Salvia miltiorrhiza Bunge (European Patent No. 568,001). Salvia extracts have also been prepared as anti-herpes virus agents (U.S. Pat. No. 5,411,733).

Several forms of salvianolic acid (e.g., salvianolic acid A and acetylsalvianolic acid) have been described as having antioxidant properties (Lin et al., J Biochem. Pharmacol. 51: 1237-1241 (1996). Salvianolic acid has also been indicated for preventing liver injury and fibrosis, associated with its anti-lipid peroxidation actions (Hu et al., Acta Pharmacol. Sin. 18: 478-480 (1997)) and for use in treating coronary diseases (Japanese Patent No. 2,131,423). Additional forms of salvianolic acid described in the literature include those isolated from aqueous extracts of Salvia cavaleriei (e.g., salvianolic acids A, B, C H and I) (Zhang et al., Planta Med. 60: 70-72 (1994)) or from S. miltiorrhiza (e.g., salvianolic acid K, a caffeic acid trimer) (Kasimu et al., Chem. Pharm. Bull. 46: 500-504 (1998); and Tezuka et al., Chem Pharm. Bull. 46: 107-112 (1998)). Salvianolic acids F 2 and F 3 can be prepared synthetically as described by Dalla et al., Tetrahedron 55: 6923-6930 (1999); and Dalla et al., Tetrahedron Lett. 39: 8285-8286 (1998). Additional members of the Salvia family may be used as sources for obtaining Danshen, including: S. bowleyana, S. deserta, S. miltiorhiza var. miltiorhiza f. alba, S. paramiltiorhiza, S. paramiltiorhiza f. purpureo-rubra, S. przewalskii, S. prsewalskii var. mandarinorum, S. sinica f. purpurea, and S. trijuga) (Kasiumu et al., 1998). Methods of producing plants with elevated secondary metabolite levels of compounds such as salvianolic acid have also been described. See U.S. Pat. No. 5,869,340 (1999). In various aspects, salvianolic acid A and derivatives thereof, are used in the methods of the disclosure as the inhibitor of PRMT.

Adenosine dialdehyde (ADOX or AdOx) is an indirect methyltransferase inhibitor broadly used to accumulate methyl-accepting proteins in hypomethylated states for protein methylation analyses. ADOX is an adenosine analog and S-adenosylmethionine-dependent methyltransferase inhibitor. ADOX inhibits S-adenosyl-L-homocystein hydrolase, resulting in the accumulation of S-adenosyl-L-homo cystein (Adoicy), a product inhibitor of methyltransferases that utilize S-adenosyl-L-methionine (AdoMet) as the methyl group donor. ADOX inhibited the Tax-activated NF-κB pathway, resulting in reactivation of p53 and induction of p53 target genes. Analysis of the NF-κB pathway demonstrated that ADOX treatment resulted in degradation of the IκB kinase complex and inhibition of NF-κB through stabilization of the NF-κB inhibitor IκBa. ADOX induced G2/M cell cycle arrest and cell death in HTLV-1-transformed but not control lymphocytes (Dasgupta, et al. J [symbol] 59). In various aspects, ADOX and derivatives thereof, are used in the methods of the disclosure as the inhibitor of PRMT.

In some embodiments, the disclosure includes a composition comprising a small molecule inhibitor of PRMT, as described herein, in combination with a pharmaceutically acceptable carrier. In various aspects, such a composition comprises or also comprises other ingredients, such as a diluent(s), excipient(s), and/or adjuvant(s). Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

The disclosure also includes a composition, as described herein, comprising any small molecule inhibitor of PRMT described herein alone or in combination with another small molecule inhibitor of PRMT, or in combination with another therapy known for treating a MD including, but not limited to, FSHD.

Sterile injectable solutions are prepared by incorporating a small molecule inhibitor of PRMT in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure provides a method of inhibiting arginine methylation of a double homeobox 4 (DUX4) protein in a cell comprising contacting the cell with an effective amount of at least one inhibitor of protein arginine methyl transferase (PRMT), wherein the inhibitor of PRMT is at least one of Compound I and Compound II, or a salt, hydrate, or stereoisomer thereof:

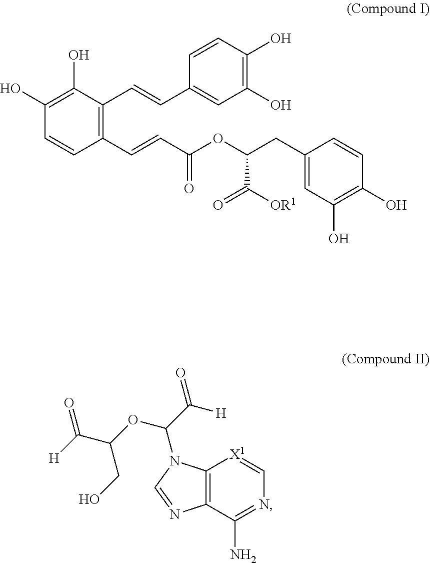

wherein R¹ is H or C₁-C₆ alkyl; and X¹ is CH or N.

The disclosure provides a method of decreasing double homeobox 4 (DUX4)-associated apoptotic cell death and/or decreasing DUX4 target gene activation in a cell comprising contacting the cell with an effective amount of at least one of Compound I and Compound II, or a salt, hydrate, or stereoisomer thereof:

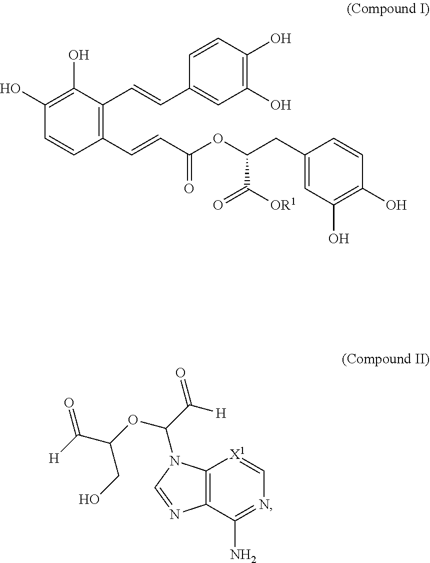

wherein R¹ is H or C₁-C₆ alkyl; and X¹ is CH or N.

The disclosure provides a method of treating an arginine methyl transferase (RMT) disorder in a patient in need thereof comprising administering to the patient a therapeutically effective amount of at least one of Compound I and Compound II, or a salt, hydrate or stereoisomer thereof:

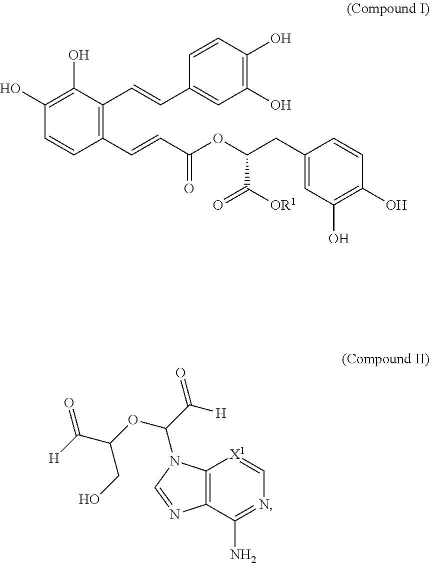

wherein R¹ is H or C₁-C₆ alkyl; and X¹ is CH or N.

In some aspects, the cell is in a subject at risk of or suffering from a muscular dystrophy. In some aspects, the subject is a human subject.

In some aspects, the PRMT disorder is a muscular dystrophy. In some aspects, the muscular dystrophy is a facioscapulohumeral muscular dystrophy (FSHD).

In some aspects, Compound I is Compound Ia:

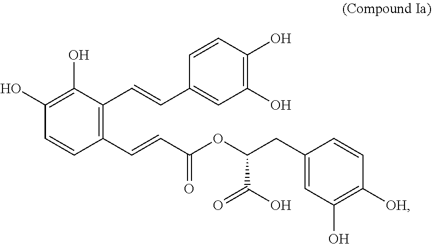 [SMILES: O=C(/C=C/c1ccc(O)c(O)c1/C=C/c1ccc(O)c(O)c1)O[C@H](Cc1ccc(O)c(O)c1)C(=O)O]

or a salt, a hydrate, or a stereoisomer thereof.

In some aspects, Compound Ia is a hydrate of formula:

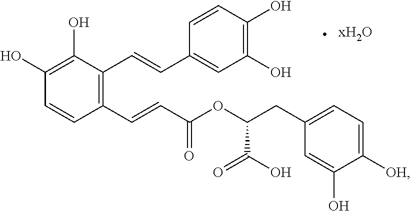 [SMILES: O=C(/C=C/c1ccc(O)c(O)c1/C=C/c1ccc(O)c(O)c1)O[C@H](Cc1ccc(O)c(O)c1)C(=O)O]

and x is 0.5 to 10.

In some aspects, Compound II is Compound IIa:

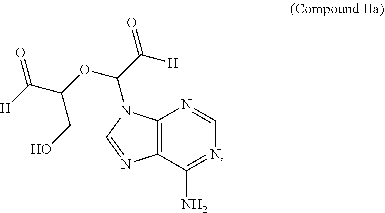 [SMILES: Nc1ncnc2c1ncn2C(C=O)OC(C=O)CO]

or a salt, a hydrate, or a stereoisomer thereof.

In some embodiments, the disclosure provides methods of inhibiting protein arginine methylation of a double homeobox 4 (DUX4) protein in a cell, decreasing double homeobox 4 (DUX4)-associated apoptotic cell death and/or decreasing DUX4 target gene activation in a cell, treating an arginine methyl transferase disorder, or treating a muscular dystrophy or cancer comprising using compounds obtained from the extracts of the plant genus Salvia (e.g., Salvia miltiorrhiza).

In some embodiments, the compounds comprise Compound A or a salt, a hydrate, or a stereoisomer thereof and/or Compound B, or a salt, a hydrate, or a stereoisomer thereof:

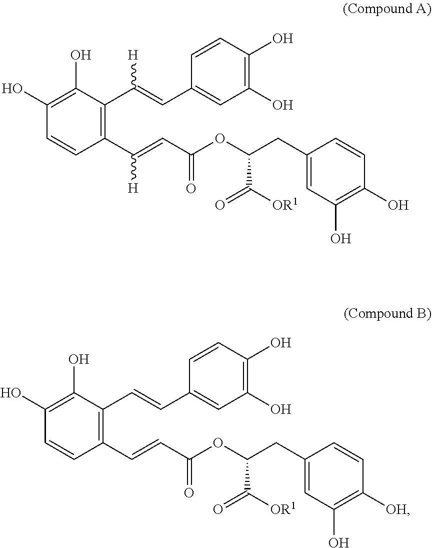

wherein R¹ is H or C₁-C₆ alkyl.

In some embodiments, the compound is salvianolic acid or a derivative thereof. For example, in some embodiments, the compound is Compound I, or a salt, a hydrate, or a stereoisomer thereof:

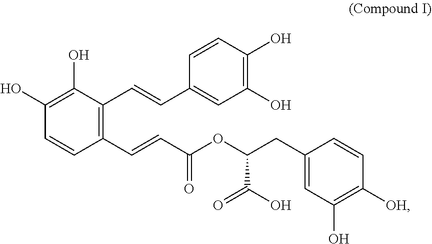

wherein R¹ is H or C₁-C₆ alkyl. In some embodiments, R¹ is H.

The compounds described herein can exist in free form, or, where appropriate, as salts. Those salts that are pharmaceutically acceptable are of particular interest since they are useful in administering the compounds described below for medical purposes. Salts that are not pharmaceutically acceptable are useful in manufacturing processes, for isolation and purification purposes, and in some instances, for use in separating stereoisomeric forms of the compounds described herein or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutica. [symbol] salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N*(C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further [symbol] acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that this disclosure includes mixtures/combinations of different pharmaceutically acceptable salts and also mixtures/combinations of compounds in free form and pharmaceutically acceptable salts.

In some embodiments, the compound is Compound Ia (i.e., salvianolic acid A (SAA)):

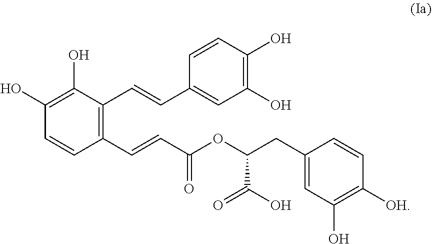 [SMILES: O=C(/C=C/c1ccc(O)c(O)c1/C=C/c1ccc(O)c(O)c1)O[C@H](Cc1ccc(O)c(O)c1)C(=O)O]

In some embodiments, the compound is a hydrate of Compound Ia of formula:

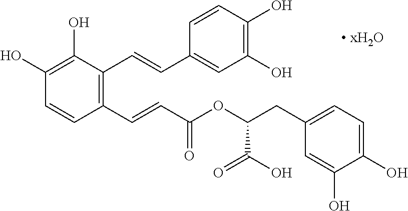

wherein x is 0.5 to 10.

As used herein, the term “hydrate” refers to the chemical entity formed by the interaction of water and a compound, including, for example, hemi-hydrates, monohydrates, dihydrates, trihydrates, etc.

In some embodiments, the compound is Compound II, or a salt, hydrate, or stereoisomer thereof:

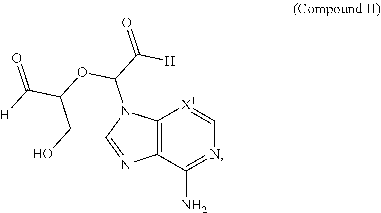

wherein X¹ is CH or N. In some embodiments, X¹ is N, such that Compound II is Compound IIa, or a salt, hydrate, or stereoisomer thereof:

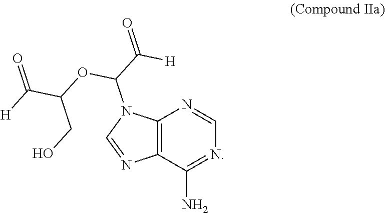 [SMILES: Nc1ncnc2c1ncn2C(C=O)OC(C=O)CO]

In some embodiments, the disclosure provides a method of inhibiting a protein arginine methyl transferase (PRMT or RMT) comprising contacting the PRMT with an effective amount of at least one of Compound I and Compound II, or a salt, a hydrate, or a stereoisomer thereof.

In some embodiments, the disclosure provides a method decreasing DUX4-induced apoptosis or cell death and/or DUX4-induced transactivation or gene expression comprising contacting the DUX4 protein with an effective amount of at least one of Compound I and Compound II, or a salt, a hydrate, or a stereoisomer thereof. In some aspects, the DUX4 protein is in a cell. In some aspects, the DUX4 protein is in a cell of a subject. In some aspects, the cell is in a human subject. In some aspects, the human subject is suffering from or is at risk of suffering from a muscular dystrophy. [symbol] aspects, the muscular dystrophy is facioscapulohumeral muscular dystrophy (FSHD).

In some embodiments, the disclosure provides a method of treating a protein arginine methyl transferase (PRMT or RMT) disorder in a patient comprising administering to the patient a therapeutically effective amount of at least one of Compound I and Compound II, or a salt, a hydrate, or a stereoisomer thereof. In some embodiments, the PRMT disorder is a muscular dystrophy including, but not limited to, FSHD.

In some embodiments, in conjunction with other above or below embodiments, the disclosed methods comprise using Compound Ia, or a salt, hydrate, or stereoisomer thereof.

In some embodiments, the disclosed methods comprise using a hydrate of Compound Ia.

In some embodiments, in conjunction with other above or below embodiments, the disclosed methods comprise using Compound II, or a salt, hydrate, or stereoisomer thereof.

In some embodiments, the disclosed methods comprise using Compound IIa, or a salt, hydrate, or stereoisomer thereof.

In some aspects, DUX4-induced apoptosis or cell death and/or DUX4-induced transactivation is decreased in a cell or in a subject by the methods provided herein by at least or about 5, about 10, about 15, about 20, about 25, about 30, about 35, about 40, about 45, about 50, about 55, about 60, about 65, about 70, about 75, about 80, about 85, about 90, about 95, about 96, about 97, about 98, about 99, or 100 percent.

The methods comprise the step of administering an effective dose, or effective multiple doses, of a composition comprising a small molecule inhibitor of PRMT of the disclosure to a subject, including an animal (such as a human being) in need thereof. If the dose is administered prior to development of the muscular dystrophy, the administration is prophylactic. If the dose is administered after the development of the muscular dystrophy, the administration is therapeutic. In embodiments of the disclosure, an effective dose is a dose that alleviates (eliminates or reduces) at least one symptom associated with the muscular dystrophy being treated, that slows or prevents progression of the muscular dystrophy, that slows or prevents progression of the muscular dystrophy, that diminishes the extent of disease, that results in remission (partial or total) of the muscular dystrophy, and/or that prolongs survival. In some aspects, the muscular dystrophy is FSHD.

Combination therapies are also contemplated by the disclosure. Combination as used herein includes simultaneous treatment or sequential treatments. Combinations of methods of the disclosure with standard medical treatments (e.g., corticosteroids and/or immunosuppressive drugs) or with other inhibitory RNA constructs are specifically contemplated, as are combinations with other therapies such as those disclosed in International Publication No. WO 2013/016352, which is incorporated by [symbol] its entirety.

Administration of an effective dose of the small molecule inhibitor of PRMT or a composition comprising the small molecule inhibitor of PRMT may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. Route(s) of administration may be chosen and/or matched by those skilled in the art taking into account the disease state being treated and the target cells/tissue(s), such as cells that express DUX4. In some embodiments, the route of administration is intramuscular. In some embodiments, the route of administration is intravenous. In some aspects, an effective dose is delivered by a systemic route of administration, i.e., systemic administration. Systemic administration is a route of administration into the circulatory system so that the entire body is affected. Such systemic administration, in various aspects, takes place via enteral administration (absorption of the drug through the gastrointestinal tract) or parenteral administration (generally via injection, infusion, or implantation). In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration, in various aspects, are chosen and/or matched by those skilled in the art taking into account the condition or state of the disease or disorder being treated, the condition, state, or age of the subject, and the target cells/tissue(s) being targeted with the small molecule inhibitor of PRMT or a composition comprising the small molecule inhibitor of PRMT.

In some aspects, actual administration of a small molecule inhibitor of PRMT or composition comprising a small molecule inhibitor of PRMT of the disclosure may be accomplished by using any physical method that will transport the small molecule inhibitor of PRMT into the target tissue of the subject, i.e., a human subject or an animal subject. Administration according to the disclosure includes, but is not limited to, injection into muscle, the bloodstream, the central nervous system, and/or directly into the brain, liver, or other organ. Simply resuspending a small molecule inhibitor of PRMT in phosphate buffered saline has been demonstrated to be sufficient to provide a vehicle useful for muscle tissue expression, and there are no known restrictions on the carriers or other components that can be co-administered with the small molecule inhibitor of PRMT. Pharmaceutical compositions can be prepared as injectable formulations or as topical formulations to be delivered to the muscles by transdermal transport. Numerous formulations for both intramuscular injection and transdermal transport have been previously developed and can be [symbol] of the disclosure. The small molecule inhibitor of PRMT can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of the small molecule inhibitor of PRMT or a pharmacologically acceptable salt can be prepared in water suitably mixed with a surfactant such as hydroxpropylcellulose. A dispersion of the small molecule inhibitor of PRMT can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. In some aspects, proper fluidity is maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, sugars, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

Sterile injectable solutions are prepared by incorporating the small molecule inhibitor of PRMT in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure provides methods of administering an effective dose (or doses, administered essentially simultaneously or doses given at intervals) of the small molecule inhibitor of PRMT designed to decrease DUX4-induced apoptosis or cell death and/or DUX4-activated gene expression (or DUX4 target gene activation or transactivation) to a cell or to a subject in need thereof. In some aspects, the effective dose is therefore a therapeutically effective dose. A therapeutically effective dose is determined by a clinician or a team of clinicians depending on the age, sex, height, weight, and condition of the subject.

In some aspects, an initial dose is followed by a second greater dose. In some aspects, an initial dose is followed by a second same dose. In some aspects, an initial dose is followed by one or more lesser doses. In some aspects, an initial dose is followed by multiple doses which are the same or greater doses.

The in vivo methods comprise the step of administering an effective dose, or effective multiple doses, of a small molecule inhibitor of PRMT or a composition comprising a small molecule inhibitor of PRMT, as described herein, to a subject (including a human subject) in need thereof. Thus, methods are provided of administering an effective dose (or doses, administered essentially simultaneously or doses given at intervals) of a small molecule inhibitor of PRMT or a composition comprising a small molecule inhibitor of PRMT described herein to a subject in need thereof. If the dose or doses is administered prior to development of a disorder/disease, the administration is prophylactic. If the dose or doses is administered after the development of a disorder/disease, the [symbol] An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

In some embodiments, compositions and methods of the disclosure are used in treating, ameliorating, or preventing a disease, such as a muscular dystrophy (MD). In various aspects, such MD is FSHD. FSHD is among the most commonly inherited muscular dystrophies, estimated to affect as many as 870,000 individuals. Classical descriptions of FSHD presentation include progressive muscle weakness in the face, shoulder-girdle and arms, but disease can manifest more broadly, including in muscles of the trunk and lower extremities. Variability is also commonly seen within individuals, as asymmetrical weakness is common. Age-at-onset can range from early childhood to adulthood, and is usually related to disease severity, where earlier onset is often associated with more severe muscle weakness. Although most patients with FSHD have a normal life span, respiratory insufficiency can occur, and the disease can be debilitating, as approximately 25% of affected individuals may become wheelchair dependent by their fifties, and even earlier in more severe forms of the disease, while others maintain lifelong ambulation.

FSHD is caused by aberrant expression of the double homeobox 4 gene (DUX4), which produces a transcription factor that is toxic to skeletal muscle. DUX4 is normally functional during the two-cell stage of human development but repressed thereafter in essentially all other tissues, except perhaps the testes. In skeletal muscles of people with FSHD, specific genetic and epigenetic factors conspire to permit DUX4 de-repression, where it then initiates several aberrant gene expression cascades, including those involved in differentiation abnormalities, oxidative stress, inflammatory infiltration, cell death and muscle atrophy.

In families known to carry pathological FSHD, the methods of the disclosure, in various aspects, are methods of preventing disease and they are carried out before the onset of disease. In other various aspects, the methods of the disclosure are carried out after diagnosis and, therefore, are methods of treating or ameliorating disease. Thus, a small molecule inhibitor of PRMT, as described herein, and compositions comprising a small molecule inhibitor of PRMT, as described herein, are used in inhibiting DUX4-induced cell death and DUX4-induced target gene expression and DUX4 signalling activated by arginine methylation in the treatment, amelioration, or prevention of a muscular dystrophy associated with DUX4 overexpression, such as FSHD.

In some embodiments, compositions and methods of the [symbol] treating, ameliorating, or preventing a disease, such as a cancer. DUX4 has been shown to be activated in some cancer types, where it functions to mask tumor cells from the immune system (Chew et al., Dev. Cell 2019 Sep. 9; 50(5):658-71). For example, DUX4 protein fusions are known to cause cancer, such as rhabdomyosarcoma and Ewing's sarcoma. A CIC-DUX4 gene fusion induces sarcomas and drives sarcoma metastasis (Yoshimoto et al., Cancer Res. 2017 Jun. 1; 77(11): 2927-2937; Okimoto et al., J Clin Invest. 2019; 129(8):3401-3406). Thus, a small molecule inhibitor of PRMT, as described herein, and compositions comprising a small molecule inhibitor of PRMT, as described herein, are used in inhibiting DUX4-induced cell death and DUX4-induced gene expression and DUX4 signalling activated by arginine methylation in the treatment, amelioration, or prevention of cancer.

Molecular, biochemical, histological, and functional outcome measures demonstrate the therapeutic efficacy of the products and methods disclosed herein (1) for decreasing the apoptosis or cell death, (2) for decreasing DUX4-induced gene expression or transactivation after arginine methylation of the DUX4 protein, and (3) for treating muscular dystrophies, such as FSHD. Outcome measures are described, for example, in Chapters 32, 35 and 43 of Dyck and Thomas, Peripheral Neuropathy, Elsevier Saunders, Philadelphia, PA, 4^{th} Edition, Volume 1 (2005) and in Burgess et al., Methods Mol. Biol., 602: 347-393 (2010). Outcome measures include, but are not limited to, reduction or elimination of DUX4-induced cell death or gene activation in affected cells and tissues. The inhibition of the arginine methylation of DUX4 in the cell is detected by methods known in the art including, but not limited to, the methods described herein, including in the Examples, before and after administration of the small molecule inhibitor of PRMT or a composition comprising the small molecule inhibitor of PRMT to determine the improvement.

In some embodiments, the methylation of DUX4 protein in a cell of the subject is decreased after administration of the small molecule inhibitor of PRMT or a composition comprising the small molecule inhibitor of PRMT as compared to the methylation of DUX4 protein before administration of the small molecule inhibitor of PRMT or a composition comprising the small molecule inhibitor of PRMT. In some aspects, methylation of DUX4 protein is decreased by at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%. In various aspects, improved muscle strength, improved muscle function, and/or improved mobility and stamina show an improvement by at least about 2%, at least about 5%, at least about 10%, at least about 20%, at least about 30%, at least about 40%, at least about 50%, at least [symbol] least about 70%, at least about 80%, at least about 90%, at least about 95%, at least about 98%, at least about 99%, at least about 100% percent, or at least about greater than 100%.

In some embodiments, apoptosis or cell death of the cells of the subject is decreased after administration of the small molecule inhibitor of PRMT or a composition comprising the small molecule inhibitor of PRMT as compared to apoptosis or cell death of the cells of the subject before administration of the small molecule inhibitor of PRMT or a composition comprising the small molecule inhibitor of PRMT. In some aspects, apoptosis or cell death of the cells of the subject is decreased by at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%. In various aspects, improved muscle strength, improved muscle function, and/or improved mobility and stamina show an improvement by at least about 2%, at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%.

Other outcome measures include measuring the level of serum creatinine kinase (CK) in the subject before and after treatment. Increased CK levels are a hallmark of muscle damage. In muscular dystrophy patients, CK levels are significantly increased above the normal range (10 to 100 times the normal level since birth). When elevated CK levels are found in a blood sample, it usually means muscle is being disintegrated by some abnormal process, such as a muscular dystrophy or inflammation. Thus, a positive therapeutic outcome for treatment with the methods of the disclosure is a reduction in the level of serum creatinine kinase after administration of the small molecule inhibitor of PRMT or a composition comprising the small molecule inhibitor of PRMT as compared to the level of serum creatinine kinase before administration of the small molecule inhibitor of PRMT or a composition comprising the small molecule inhibitor of PRMT.

Other outcome measures include measuring to determine if there is improved muscle strength, improved muscle function, improved mobility, improved stamina, or a combination of two or more thereof in the subject after treatment. Such outcome measures are important in determining muscular dystrophy progression in the subject and are measured by various tests known in the art. Some of these tests include, but are not limited to, the six minute walk test, time to rise test, ascend 4 steps test, ascend and descend 4 steps test, North Star Ambulatory Assessment (NSAA) test, 10 meter timed test, 100 meter timed test, hand held dynamometry (HHD) test, Timed Up and Go test, [symbol] Scaled (Bayley-III) score, maximum isometric voluntary contraction test (MVICT), or a combination of two or more thereof.

Combination therapies are also included the disclosure. Combination, as used herein, includes both simultaneous treatment and sequential treatments. Combinations of methods described herein with standard medical treatments and supportive care are specifically contemplated, as are combinations with therapies, such as glucocorticoids. All types of glucocorticoids are included for use in the combination therapies disclosed herein. Such glucocorticoids include, but are not limited to, prednisone, prednisolone, dexamethasone, deflazacort, beclomethasone, betamethasone, budesonide, cortisone, hydrocortisone, methylprednisolone, and triamcinolone.

Other combination therapies included in the disclosure are the small molecule inhibitor of PRMT or a composition comprising the small molecule inhibitor of PRMT, as described herein, in combination with a U7-snRNA, an miRNA-based gene therapy, a small molecule inhibitor of DUX4 expression, oligonucleotides to inhibit DUX4 through RNAi or RNAse H or exon skipping mechanisms, or a U7-snRNA plus a theoretical CRISPR-based gene therapy approach.

“Treating” includes ameliorating or inhibiting one or more symptoms of a muscular dystrophy including, but not limited to, muscle wasting, muscle weakness, myotonia, skeletal muscle problems, abnormalities of the retina, hip weakness, facial weakness, abdominal muscle weakness, joint and spinal abnormalities, lower leg weakness, shoulder weakness, hearing loss, muscle inflammation, and nonsymmetrical weakness.

The disclosure also provides a kit comprising an inhibitor of PRMT or a composition comprising an inhibitor of PRMT of the disclosure. In the context of the disclosure, the term “kit” means two or more components, one of which corresponds to an inhibitor of PRMT or a composition comprising an inhibitor of PRMT of the disclosure, and the other which corresponds to a container, recipient, instructions, or otherwise. A kit, therefore, in various aspects, is a set of products that are sufficient to achieve a certain goal, which can be marketed as a single unit.

The kit may comprise one or more recipients (such as vials, ampoules, containers, syringes, bottles, bags) of any appropriate shape, size and material containing an inhibitor of PRMT or a composition comprising an inhibitor of PRMT of the disclosure in an appropriate dosage for administration (see above). The kit may additionally contain directions or instructions for use (e.g. in the form of a leaflet or instruction manual), means for administering an inhibitor of PRMT or a composition comprising an inhibitor of PRMT, such as a syringe, pump, infuser or the like, means for reconstituting the [symbol] or a composition comprising the inhibitor of PRMT and/or means for diluting the inhibitor of PRMT or a composition comprising the inhibitor of PRMT.

In some aspects, the kit comprises a label and/or instructions that describes use of the reagents provided in the kit. The kits also optionally comprise catheters, syringes or other delivering devices for the delivery of one or more of the compositions used in the methods described herein.

The disclosure also provides kits for a single dose of administration unit or for multiple doses. In some embodiments, the disclosure provides kits containing single-chambered and multi-chambered pre-filled syringes.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning.

Unless otherwise indicated, the term “at least” preceding a series of elements is to be understood to refer to every element in the series. Those skilled in the art will recognize, or be able to ascertain using no more than routine experimentation, many equivalents to the specific embodiments of the disclosure described herein. Such equivalents are intended to be encompassed by the disclosure.

The term “and/or” wherever used herein includes the meaning of “and”, “or” and “all or any other combination of the elements connected by said term.”

The term “about” or “approximately” as used herein means within 20%, preferably within 10%, and more preferably within 5% of a given value or range. It includes, however, also the concrete number, e.g., about 10 includes 10.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the [symbol] or “including” or sometimes when used herein with the term “having.”

When used herein, “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this disclosure is not limited to the particular methodology, protocols, material, reagents, and substances, etc., described herein and as such can vary. The terminology used herein is for the purpose of describing particular embodiments only, and is not intended to limit the scope of the subject matter of the disclosure, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material.

A better understanding of the disclosure and of its advantages will be obtained from the following examples, offered for illustrative purposes only. The examples are not intended to limit the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

### Materials and Methods

### Mutagenesis

DUX4 modification mutant plasmids (single, double, triple, HOX1_methyl-null, basic and HOX1_methyl-mimic mutant constructs) were constructed using a recombinant PCR method. The mutation was constructed using PCR to amplify the DUX4 ORF, with primers containing the mutated site, using CMV driven wildtype DUX4 [symbol] (AAV.DUX4.V5) as the template. The entire mutant DUX4 ORF was amplified, gel purified and cloned into PCR-blunt II-topo, prior to sequence verification. The DUX4 mutant ORF was then cloned into AAV.CMV.DUX4 or AAV.CMV.eGFP to replace either wild-type DUX4 or eGFP, respectively. Phospho-null, Phospho-mimic, Methyl-null, basic and Methyl-mimic mutants were synthesized by Genscript with N-terminal NheI and C-terminal Acc651 restriction enzyme sites flanking the DUX4 ORF using AAV.CMV.eGFP as the base vector.

### Cell Culture

Human embryonic kidney cells (HEK293) and human immortalized myoblasts (WS236, 15V, biceps, unaffected control cells) were maintained as previously described⁹. Briefly, HEK293 cells were cultured in DMEM supplemented with 10% fetal bovine serum, L-glutamine and penicillin/streptomycin at 37° C. in 5% CO₂. Human immortalized myoblasts were cultured in LHCN media containing DMEM supplemented with 16% Medium 199, 15% fetal bovine serum, 30 ng/ml zinc sulfate, 1.4 μg/ml vitamin B12, 55 ng/ml dexamethasone, 2.5 ng/ml human growth factor, 10 ng/ml fibroblast growth factor, 20 mM HEPES and penicillin/streptomycin.

### Protein Immunoprecipitation

HEK293 cells were transfected in suspension with a total of 4 μg AAV.CMV.DUX4.V5, PRMT1-GFP, or PRKACA-Flag (1×10⁶ cells/well) using Lipofectamine 2000 and harvested 16 hours later using cold 1×PBS. Cells were pelleted and lysed in Buffer A containing 137 mM NaCl, 50 mM Tris, pH 7.5, 1% NP-40, protease inhibitor cocktail (Sigma), and phosphatase inhibitors including sodium pyrophosphate, p-glycerol phosphate, sodium fluoride and sodium orthovanadate. All steps were performed at 4° C. or on ice. Lysate was incubated with protein agarose G for 1 hour, while rotating. The supernatant was subsequently incubated with anti-V5 antibody conjugated to agarose resin overnight, while rotating. The resin was washed five times with Buffer A, then resuspended in Buffer A supplemented with 1 mM DTT and 1×LDS-PAGE (Invitrogen) sample buffer. DUX4 complexes were eluted by boiling at 95° C. for 10 min.

### High Resolution Mass Spectrometry Sample Preparation and Peptide Digestion

Immunoprecipitated protein samples were loaded into a TGX 4-15% precast gel (Bio-Rad), resolved and stained with Bio-Safe Coomassie (Bio-Rad). The band corresponding to DUX4 was excised. Potential disulfide bonds were reduced and alkylated. Gel pieces were subjected to overnight digestion at 37° C. with 800 ng of trypsin (Promega) and/or chymotrypsin (Promega) in 100 mM ammonium bicarbonate (Sigma). Peptides were extracted from the gel matrix, dried by vacuum centrifugation and [symbol] buffer (2% acetonitrile, 0.1% formic acid).

### Liquid Chromatography and Mass Spectrometry (LC-MS/MS)

Peptides were separated on a Thermo Dionex UltiMate 3000 RSLC HPLC system coupled to a Thermo Orbitrap Fusion Tribrid mass spectrometer. Peptides were loaded on a PepMap100 C18 microcolumn (5 m, 100 Å, 0.3×50 mm) and desalted for four minutes with 0.5% TFA in 2% acetonitrile. Mobile phase solvents were buffer A: 0.1% formic acid in water and buffer B: 0.1% formic acid in acetonitrile. Peptides were eluted along a linear gradient (5-30%) of buffer B at a flow rate of 300 nL/min over 140 min followed by a column wash and equilibration. Peptide separation was performed on a Thermo EASY-Spray PepMap C18 column (3 m, 100 Å, 0.75×150 mm) operated at 275° C. and a spray voltage of 1.7 kV.

MS/MS data were collected on the Orbitrap Fusion operated in top-speed mode with a 3 s cycle time. MS1 scans were collected at 60 K resolution in the Orbitrap prior to HCD fragmentation at 27% normalized collision energy. Fragment ions were isolated in the quadrupole with an isolation window of 1.6 m/z and detected in the Orbitrap at 15 K resolution. AGC targets were set to 4E5 or 50 ms maximum injection time for MS1 scans and 5E4 or 500 ms maximum injection time for MS2 scans to maximize ion series coverage. Dynamic exclusion was set to ±10 ppm for a period of 30 s.

### Mass Spectrometry Data Analysis

RAW data were converted to the mxXML format using the MSConvert tool in ProteoWizard (v3.0.4624) and searched against a database containing the DUX4 sequence downloaded from UniProt (accession Q9UBX2) with a C-terminal V5 epitope tag (GKPIPNPLLGLDST (SEQ ID NO: 3)) and common contaminant proteins (downloaded 22 Jun. 2015; 234 total entries) using the MassMatrix search engine v2.4.2¹⁰⁻¹⁴. Peptide mass tolerance was set at 20 ppm with a fragment mass tolerance of 0.02 Da. The data were also searched on MASCOT (version 2.6.0, Matrix Science) against uniprot human database containing the DUX4 sequence downloaded from UniProt (accession Q9UBX2) with a C-terminal V5 epitope tag (GKPIPNPLLGLDST (SEQ ID NO: 3)). Peptide mass tolerance was set at 10 ppm with a fragment mass tolerance of 0.05 Da. For both search engine, variable modifications included acetylation of K; mono-, di- or tri-methylation of K; mono- or di-methylation of R; oxidation of M; and phosphorylation of S,T,Y. Carbamidomethylation of C was included as a fixed modification. Enzyme specificity was set for trypsin and chymotrypsin with up to four missed cleavages. Search-generated peptides that also passed manual validation were included in the calculation of sequence coverage.

RAW data were converted to the mxXML format using the [symbol] ProteoWizard (v3.0.4624) and searched against a database containing the DUX4 sequence downloaded from UniProt (accession Q9UBX2) with a C-terminal V5 epitope tag (GKPIPNPLLGLDST (SEQ ID NO: 3)) and common contaminant proteins (downloaded 22 Jun. 2015; 234 total entries) using the MassMatrix search engine v2.4.2. Peptide mass tolerance was set at 20 ppm with a fragment mass tolerance of 0.02 Da. Variable modifications included acetylation of K; mono-, di- or tri-methylation of K; mono- or di-methylation of R; oxidation of M; and phosphorylation of S,T,Y. Carbamidomethylation of C was included as a fixed modification. Enzyme specificity was set for trypsin and chymotrypsin with up to four missed cleavages. Search-generated peptides that also passed manual validation were included in the calculation of sequence coverage

### Quantitative PCR

HEK293 cells and human myoblasts (5×105 cells/well) were transfected with 2 g of AAV.DUX4.V5 (wild type or mutant construct) using Lipofectamine 2000 (Thermo Scientific). Cells were harvested 24 hours post-transfection in TRIzol RNA isolation reagent (Life Technologies). RNA was isolated, DNase treated and reverse transcribed into cDNA using the High-Capacity cDNA Reverse Transcription Kit (Applied Biosystems). TaqMan gene expression assays (Applied Biosystems) were used to quantify human RPL13A (Hs01494366_g1), human ZSCAN4 (Hs00537549_ml) and human PRAMEF12 (Hs04193637_mH). Efficiencies were comparable among all probes. RPL13A was used as the reference gene for normalization. The normalized expression (ΔΔCq) was calculated relative to control pCINeo transfected cells.

### Caspase-3/7 Activation Assay

HEK293 cells were transfected with 100 ng plasmid DNA (65,000 cells/well) using Lipofectamine 2000 and assays were performed 48 hours later using the Apo-ONE Homogeneous Caspase-3/7 Assay (Promega). The protocol was followed according to the manufacturer's instructions. Briefly, 100 μl of the reagent was added to each well and the 96-well plate was gently rotated for 20 min. Relative fluorescence was monitored every hour for 4-6 hours.

Inhibitor studies were performed in human myoblasts which were transfected with 100 ng of DUX4 DNA (65,000 cells/well) with Lipofectamine 2000. The inhibitors adenosine dialdehyde or salvianolic acid A were added 1.5 hours later and a caspase assay was performed 48 hours later as above.

### DUX4-Activated Reporter in Cells

HEK293 cells (65,000 cells/well) were transfected in suspension [symbol] plasmid DNA (AAV.CMV.DUX4.V5 or mutants) and 100 ng pLenti.DUX4-activated GFP, previously reported in (ref), using Lipofectamine 2000 (Thermo Scientific) and plated simultaneously on 96-well plates. GFP expression was quantified 24 and 48 hours post-transfection using the SPECTRAmax M2 instrument (Molecular Devices). GFP expression was visually monitored with a fluorescent stereo microscope (Leica M165 FC microscope, Leica Microsystems).

### Rapid Immunoprecipitation Mass Spectrometry of Endogenous Proteins (RIME)

Cells were fixed with 1% formaldehyde for 8 min and quenched with 0.125 M glycine. Chromatin was isolated by the addition of lysis buffer, followed by disruption with a Dounce homogenizer. Lysates were sonicated and the DNA sheared to an average length of 300-500 base-pairs. Genomic DNA (Input) was prepared by treating aliquots of chromatin with RNase, proteinase K and heat for de-crosslinking, followed by ethanol precipitation. Pellets were resuspended and the resulting DNA was quantified on a NanoDrop spectrophotometer. Extrapolation to the original chromatin volume allowed quantitation of the total chromatin yield.

An aliquot of chromatin (100 μg) was pre-cleared with protein G agarose beads (Invitrogen). Proteins of interest were immunoprecipitated using 10 μg of antibody against V5 (Abcam, ab15828) and protein G magnetic beads. Protein complexes were washed, then trypsin was used to remove the immunoprecipitate from beads and the digested protein sample. Protein digests were separated from the beads and purified using a C18 spin column (Harvard Apparatus). The peptides were vacuum dried using a speedvac.

Digested peptides were analyzed by LC-MS/MS on a Thermo Scientific Q Exactive Orbitrap Mass spectrometer in conjunction with a Proxeon Easy-nLC II HPLC (Thermo Scientific) and Proxeon nanospray source. The digested peptides were loaded on a 100 micron×25 mm Magic C18 100 Å 5U reverse phase trap where they were desalted online before being separated using a 75 micron×150 mm Magic C18 200 Å 3U reverse phase column. Peptides were eluted using a 90 minute gradient with a flow rate of 300 nl/min. An MS survey scan was obtained for the m/z range 300-1600, MS/MS spectra were acquired using a top 15 method, where the top 15 ions in the MS spectra were subjected to HCD (High Energy Collisional Dissociation). An isolation mass window of 1.6 m/z was for the precursor ion selection, and normalized collision energy of 27% was used for fragmentation. A five second duration was used for the dynamic exclusion. Tandem mass spectra were extracted. Charge state deconvolution and deisotoping were not performed. All MS/MS samples were analyzed using X! Tandem (The GPM, thegpm.org; version CYCLONE (2013.02.01.1)). X! Tandem was set up to search the uniprot-_2 [symbol] database (unknown version, 141320 entries) assuming the digestion enzyme trypsin. X! Tandem was searched with a fragment ion mass tolerance of 20 PPM and a parent ion tolerance of 20 PPM. Carbamidomethyl of cysteine was specified in X! Tandem as a fixed modification. Glu->pyro-Glu of the n-terminus, ammonia-loss of the n-terminus, gln->pyro-Glu of the n-terminus, deamidated of asparagine and glutamine, oxidation of methionine and tryptophan, dioxidation of methionine and tryptophan and acetyl of the n-terminus were specified in X! Tandem as variable modifications.

Scaffold (version Scaffold_4.6.1, Proteome Software Inc., Portland, OR) was used to validate MS/MS based peptide and protein identifications. Peptide identifications were accepted if they exceeded specific database search engine thresholds. X! Tandem identifications required at least −Log(Expect Scores) scores of greater than 1.5. Protein identifications were accepted if they contained at least 1 identified peptide. Proteins that contained similar peptides and could not be differentiated based on MS/MS analysis alone were grouped to satisfy the principles of parsimony. Proteins sharing significant peptide evidence were grouped into clusters.

Final list generation was done by taking all proteins with a spectral count of five and above from each replicate reaction and comparing them in a Venn-diagram against IgG control replicates.

### Example 2

Arginine Methylation Inhibition is Associated with Decreased Apoptotic Cell Death and Decreased DUX4 Target Gene Activation

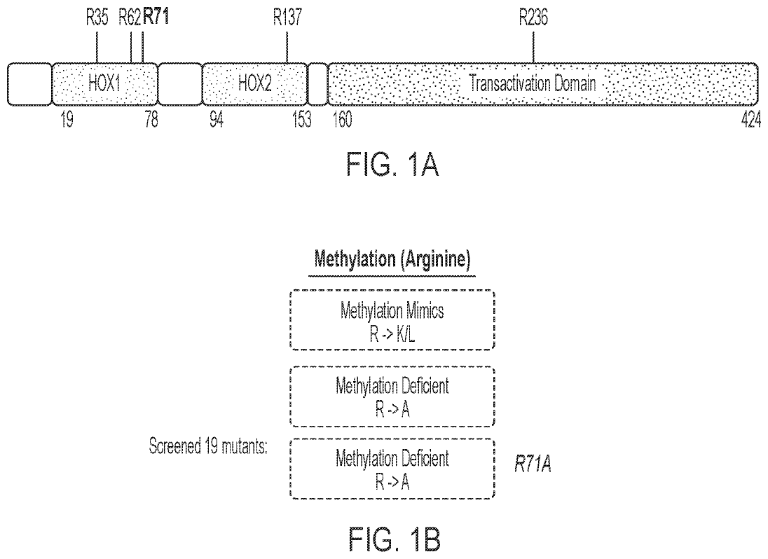

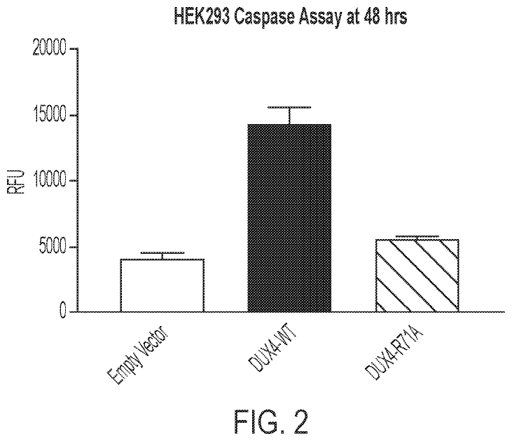

DUX4 post-translational modifications (PTMs) were detected by overexpressing DUX4 in HEK293 cells, immunoprecipitating DUX4 and performing mass spectrometry. Using this method, several DUX4 arginine methylation PTMs were identified (FIG. 1A) and methylation mimic and deficient mutants were generated to further characterize them (FIG. 1B). An arginine methylation null mutant, DUX4 R71A, was identified. The DUX4 R71A mutant protects against DUX4-mediated apoptotic cell death as indicated by decreased caspase cleavage (FIG. 2).

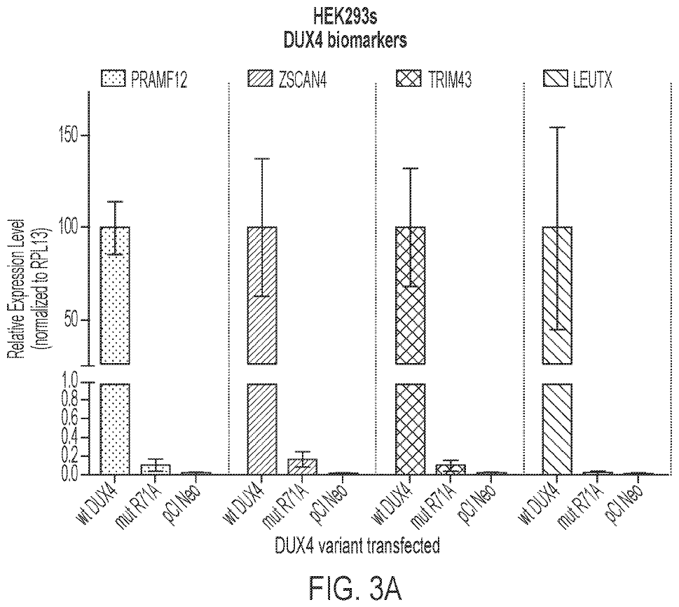

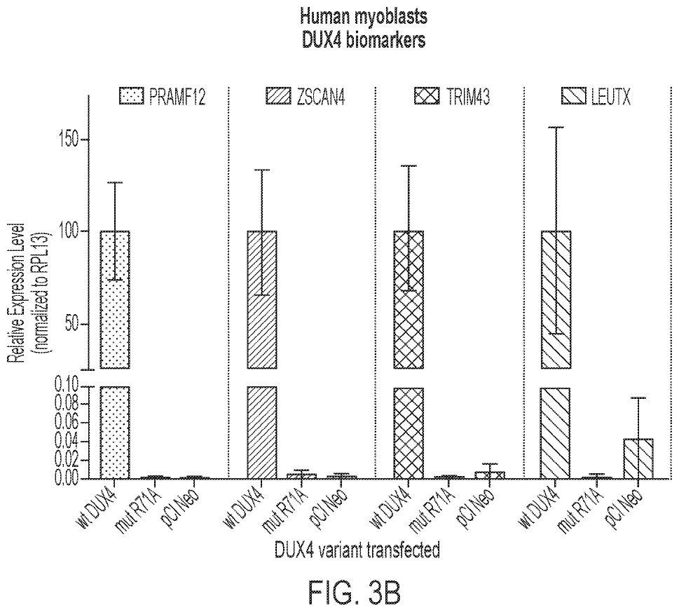

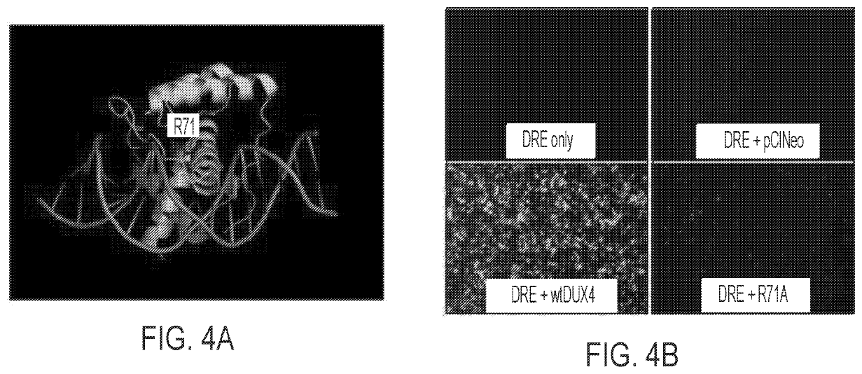

Experiments were then carried out to determine whether the arginine methylation null mutant affects DUX4's ability to activate target genes. Wild type or DUX4 R71A was expressed in HEK293 cells and in human myoblasts. DUX4 target gene expression was examined by quantitative RT-PCR. DUX4 R71A led to decreased expression of DUX4 target genes, i.e., PRAME Family Member 12 (PRAMEF12), Zinc finger and SCAN domain-containing protein 4 (ZSCAN4), Tripartite Motif Containing 43 (TRIM43), and Leucine Twenty Homeobox (LEUTX) (FIG. 3A-B). Interestingly, R71 is in close proximity to where DUX4 binds DNA (FIG. 3A). It was postulated that DUX4 R71A could interfere with [symbol] to bind DNA and, therefore, the DUX4-activated fluorescence reporter (DRE) assay⁴ was used to investigate this further. Consistent with the postulated hypothesis, DUX4 R71A is associated with decreased reporter gene expression (FIG. 4B). These results demonstrate that arginine methylation inhibition is associated with decreased apoptotic cell death and decreased DUX4 target gene activation.

### Example 3

### Arginine Methylation Inhibition Protects Against DUX4-Mediated Cell Death in Myoblasts

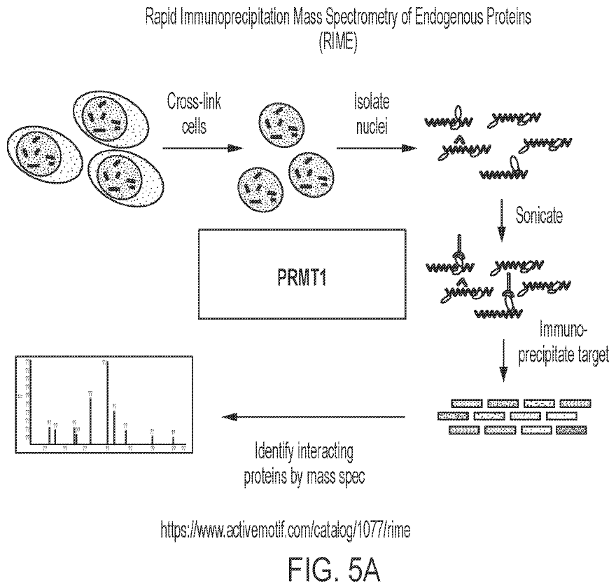

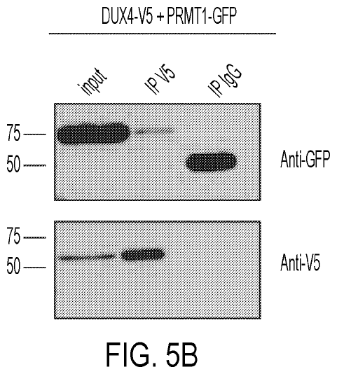

To isolate arginine methyltransferases which associate with the DUX4 complex, a proteomics approach was carried out. Rapid Immunoprecipitation Mass Spectrometry of Endogenous Proteins (RIME) combines crosslinking, immunoprecipitation and mass spectrometry to identify transient or distant interactions¹⁶. RIME was carried out in human myoblasts expressing wild type DUX4 and protein arginine methyltransferase 1 (PRMT1) as was identified as a component of the DUX4 complex (FIG. 5A). Additionally, DUX4 and PRMT1 interact when overexpressed in HEK293 cells (FIG. 5B).

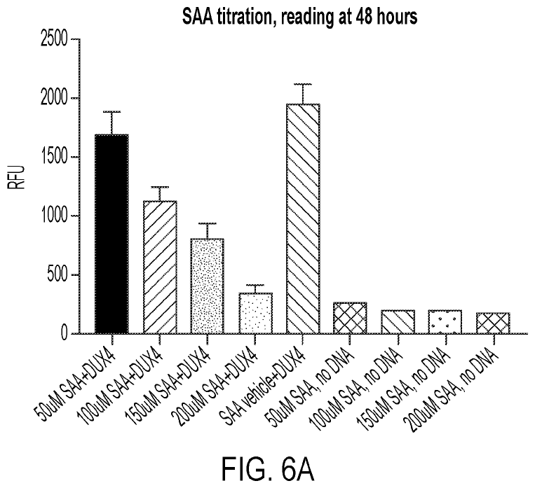

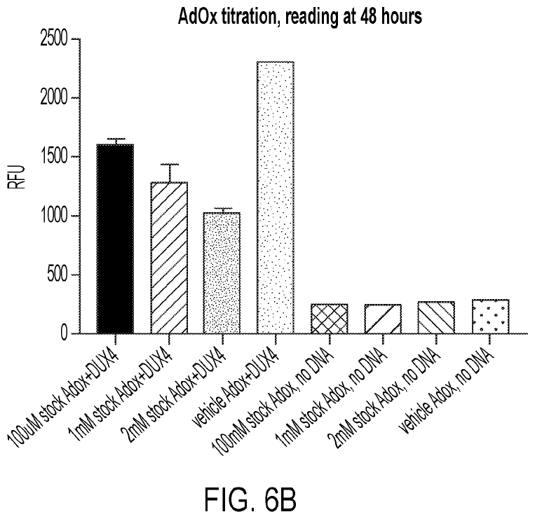

The observation that a DUX4 arginine methylation null mutant protects from cell death and DUX4 interacts with an arginine methyltransferase indicate a role for arginine methylation inhibitors as protective in FSHD disease models. Thus, the global methylation inhibitor, adenosine dialdehyde (AdOx)¹⁷, and the PRMT1 inhibitor, salvianolic acid A (SAA)¹⁸ were tested for their ability to protect against DUX4-mediated cell death. The caspase assay was performed in human myoblasts expressing DUX4 in the presence or absence of AdOx or SAA. SAA and AdOx led to decreased caspase cleavage in a dose-responsive manner (FIGS. 6A-B) indicating that arginine methylation inhibitors protect against DUX4-mediated cell death in myoblasts.

This study identifies arginine methylation as a critical regulator of DUX4-mediated toxicity. The arginine methylation null mutant DUX4 R71A leads to decreased apoptotic cell death and decreased DUX4 target gene expression. DUX4 forms a complex with arginine methyltransferase PRMT1, and arginine methylation inhibitors protect against DUX4 mediated toxicity in human myoblasts. Taken together, these results indicate that inhibition of arginine methylation is a target for FSHD therapy.

### Example 4

SAA and/or AdOx Decrease DUX4-Activated Biomarker Expression in a Mouse Model of FSHD

SAA and AdOx, or derivatives thereof, as described herein, are each injected into a FSHD mouse model (TIC-DUX4) or any other mouse model of FSHD mice intramuscularly (IM) or intravenously (IV). After 4, 8, 12, 16, 20, and 24 weeks, the [symbol] DUX4, a DUX4 biomarker, such as Wfdc3 or Trim36, and DUX4 target gene expression are measured by qRT-PCR, RNAscope, or ddPCR.

Reduced levels of DUX4 biomarker expression are observed in muscles of mice treated with SAA or AdOx compared to the levels in muscles of untreated mice. Treatment with SAA, or a derivative thereof, or AdOx, or a derivative thereof also causes decreased expression of DUX4 target genes, i.e., PRAMEF12, ZSCAN4, TRIM43, and LEUTX, in the treated mice compared to the untreated mice. These reduced levels of expression or decreased expression are associated with improved muscle strength, morphology, and overall ambulation and motor function.

### Example 5

SAA and/or AdOx Decrease DUX4-Activated Cell Death in Muscle

SAA and AdOx, or derivatives thereof, as described herein, are injected into patients suffering from FSHD intramuscularly (IM) or intravenously (IV). Prior to treatment and after 4, 8, 12, 16, 20, 24, 28, 32, 36 40, 44, 48, and 52 weeks, DUX4-activated biomarkers and/or DUX4 target genes in muscle of the patients are measured in biopsied muscle as described herein.

Reduced expression levels of DUX4-activated biomarkers and/or DUX4 target genes, i.e., PRAMEF12, ZSCAN4, TRIM43, and LEUTX, are observed in muscles or muscle cells of patients treated with SAA and AdOx, or derivatives thereof, compared to the levels of DUX4-activated biomarkers and/or DUX4 target genes of the same patients prior to treatment. Improvement in FSHD disease symptoms is also observed. Such measures of improvement include, but are not limited to, reductions in fibrosis, and improved function as measured by reachable workspace and patient reported outcomes.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist [symbol] combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment.

Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control. References referred to herein with numbering are provided with the full citation as shown herein below.

### REFERENCES

1. Hamel, J. & Tawil, R. Facioscapulohumeral Muscular Dystrophy: Update on Pathogenesis and Future Treatments. Neurotherapeutics: the journal of the American Society for Experimental NeuroTherapeutics 15, 863-871 (2018).
2. Lemmers, R. J. L. F. et al. A unifying genetic model for facioscapulohumeral muscular dystrophy. Science 329, 1650-1653 (2010).
3. Snider, L. et al. Facioscapulohumeral dystrophy: incomplete suppression of a retrotransposed gene. PLoS genetics 6, e1001181 (2010).
4. Rickard, A. M., Petek, L. M. & Miller, D. G. Endogenous DUX4 expression in FSHD myotubes is sufficient to cause cell death and disrupts RNA splicing and cell migration pathways. Human molecular genetics 24, 5901-5914 (2015).
5. Geng, L. N. et al. DUX4 activates germline genes, retroelements, and immune mediators: implications for facioscapulohumeral dystrophy. Developmental cell 22, 38-51 (2012).
6. Guccione, E. & Richard, S. The regulation, functions and clinical relevance of arginine methylation. Nat Rev Mol Cell Bio 20, 642-657 (2019).
7. Blanc, R. S. & Richard, S. Arginine Methylation: The Coming of Age. Mol Cell 65, 8-24 (2017).
8. Smith, E. et al. Recent advances in targeting protein arginine methyltransferase enzymes in cancer therapy. Expert Opin Ther Tar 22, 1-19 (2018).
9. Eidahl, J. O. et al. Mouse Dux is myotoxic and shares partial functional homology with its human paralog DUX4. Hum Mol Genet 25, 4577-4589 (2016).
10. Kessner, D., Chambers, M., Burke, R., Agus, D. & Mallick, P. ProteoWizard: open source software for rapid proteomics tools development. Bioinformatics 24, 2534-2536 (2008).
11. Craig, R., Cortens, J. P. & Beavis, R. C. Open Source System for Analyzing, Validating, and Storing Protein Identification Data. J Proteome Res 3, 1234-1242 (2004).
12. Xu, H. & Freitas, M. A. A mass accuracy sensitive probability based scoring algorithm for database searching of tandem mass spectrometry data. Bmc Bioinformatics 8, 133 (2007).
13. Xu, H., Yang, L. & Freitas, M. A. A robust linear regression based algorithm for automated evaluation of peptide identifications from shotgun proteomics by use of reversed-phase liquid chromatography retention time. Bmc Bioinformatics 9, 347 (2008).
14. Xu, H. & Freitas, M. A. Monte Carlo Simulation-Based Algorithms for Analysis of Shotgun Proteomic Data. J Proteome Res 7, 2605-2615 (2008).
15. Wallace, L. M. et al. DUX4, a candidate gene for facioscapulohumeral muscular dystrophy, causes p53-dependent myopathy in vivo. Ann Neurol 69, 540-552 (2011).
16. Mohammed, H. et al. Endogenous Purification Reveals GREB1 as a Key Estrogen Receptor Regulatory Factor. Cell Reports 3, 342-349 (2013).
17. Bartel, R. L. & Borchardt, R. T. Effects of adenosine dialdehyde on S-adenosylhomocysteine hydrolase and S-adenosylmethionine-dependent transmethylations in mouse L929 cells. Mol Pharmacol 25, 418-24 (1984).
18. Li, T. et al. Protein arginine methyltransferase 1 may be involved in pregnane x receptor-activated overexpression of multidrug resistance 1 gene during acquired multidrug resistant. Oncotarget 7, 20236-20248 (2015).

The disclosure has been described in terms of particular embodiments found or proposed to comprise specific modes for the practice of the disclosure. Various modifications and variations of the disclosure will be apparent to those skilled in the art without departing from the scope and spirit of the disclosure. Although the disclosure has been described in connection with specific embodiments, it should be [symbol] methods of the disclosure as claimed should not be unduly limited to such specific embodiments. Indeed, various modifications of the described modes for carrying out the methods that are obvious to those skilled in the relevant fields are intended to be within the scope of the following claims.

We claim:

1. A method of inhibiting arginine methylation of a double homeobox 4 (DUX4) protein in a cell comprising contacting the cell with an effective amount of at least one inhibitor of protein arginine methyl transferase (PRMT), wherein the inhibitor of PRMT is at least one of Compound I and Compound II, or a salt, hydrate, or stereoisomer thereof: 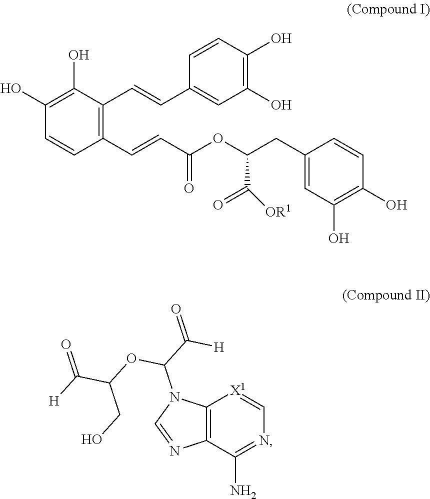
wherein R¹ is H or C₁-C₆ alkyl; and X¹ is CH or N.

2. A method of decreasing double homeobox 4 (DUX4)-associated apoptotic cell death and/or decreasing DUX4 target gene activation in a cell comprising contacting the cell with an effective amount of at least one of Compound I and Compound II, or a salt, hydrate, or stereoisomer thereof: 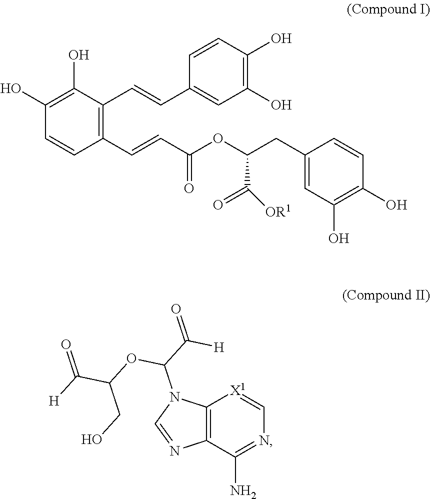
wherein R¹ is H or C₁-C₆ alkyl; and X¹ is CH or N.

3. A method of treating a protein arginine methyl transferase (PRMT) disorder in a patient in need thereof comprising administering to the patient a therapeutically effective amount of at least one of Compound I and Compound II, or a salt, hydrate or stereoisomer thereof: 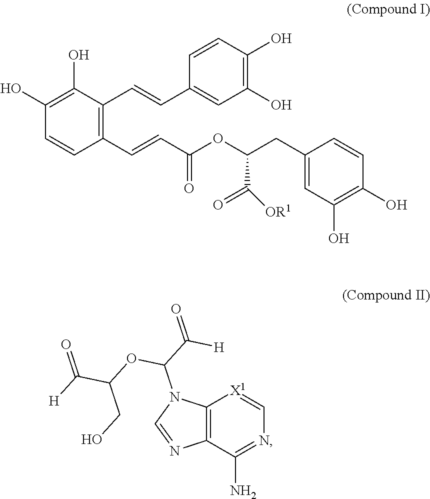
wherein R¹ is H or C₁-C₆ alkyl; and X¹ is CH or N.

4. The method of claim 1 or 2, wherein the cell is in a subject at risk of or suffering from a muscular dystrophy.

5. The method of claim 3, wherein the PRMT disorder is a muscular dystrophy.

6. The method of claim 4 or 5, wherein the muscular dystrophy is a facioscapulohumeral muscular dystrophy (FSHD).

7. The method of any one of claims 1-6, wherein Compound I is Compound Ia: 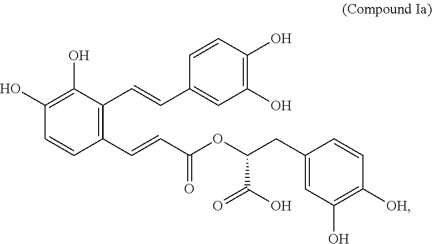 [SMILES: O=C(/C=C/c1ccc(O)c(O)c1/C=C/c1ccc(O)c(O)c1)O[C@H](Cc1ccc(O)c(O)c1)C(=O)O]
or a salt, a hydrate, or a stereoisomer thereof.

8. The method of claim 7, wherein Compound Ia is a hydrate of formula: 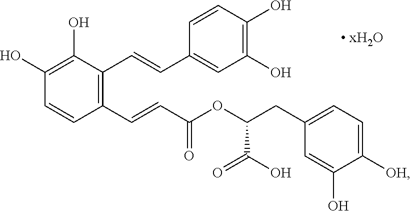 [SMILES: O=C(/C=C/c1ccc(O)c(O)c1/C=C/c1ccc(O)c(O)c1)O[C@H](Cc1ccc(O)c(O)c1)C(=O)O]
and x is 0.5 to 10.

9. The method of any one of claims 1-6, wherein Compound II is Compound IIa: 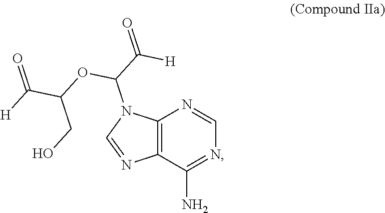 [SMILES: Nc1ncnc2c1ncn2C(C=O)OC(C=O)CO]
or a salt, a hydrate, or a stereoisomer thereof.

10. Use of at least one of Compound I and Compound II, or a salt, hydrate, or stereoisomer thereof: 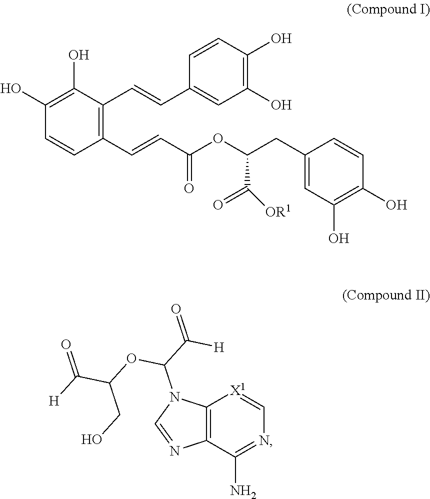
wherein R¹ is H or C₁-C₆ alkyl; and X¹ is CH or N for the preparation of a medicament for inhibiting protein arginine methylation of a double homeobox 4 (DUX4) protein in a cell.

11. Use of at least one of Compound I and Compound II, or a salt, hydrate, or stereoisomer thereof: 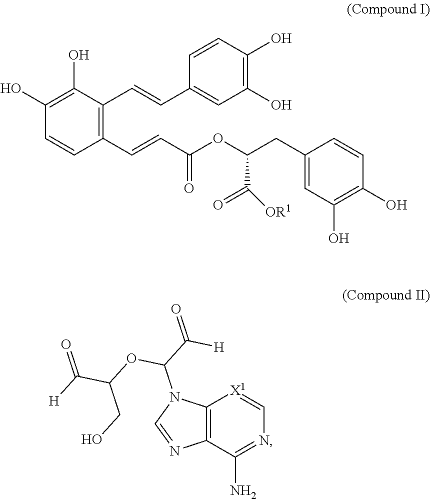
wherein R¹ is H or C₁-C₆ alkyl; and X¹ is CH or N for the preparation of a medicament for decreasing double homeobox 4 (DUX4)-associated apoptotic cell death and/or decreasing DUX4 target gene activation in a cell.

12. Use of at least one of Compound I and Compound II, or a salt, hydrate, or stereoisomer thereof: 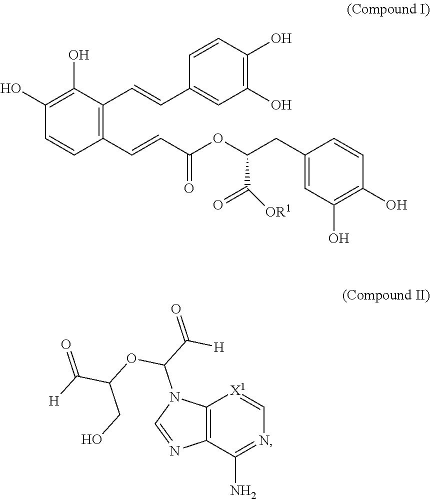
wherein R¹ is H or C₁-C₆ alkyl; and X¹ is CH or N for the preparation of a medicament for treating a protein arginine methyl transferase (PRMT) disorder in a patient in need thereof.

13. The use of claim 10 or 11, wherein the cell is in a subject at risk of or suffering from a muscular dystrophy.

14. The use of claim 12, wherein the PRMT disorder is a muscular dystrophy.

15. The use of claim 13 or 14, wherein the muscular dystrophy is a facioscapulohumeral muscular dystrophy (FSHD).

16. The use of any one of claims 10-15, wherein Compound I is Compound Ia: 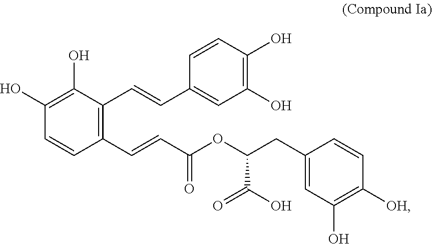 [SMILES: O=C(/C=C/c1ccc(O)c(O)c1/C=C/c1ccc(O)c(O)c1)O[C@H](Cc1ccc(O)c(O)c1)C(=O)O]
or a salt, a hydrate, or a stereoisomer thereof.

17. The use of claim 16, wherein Compound Ia is a hydrate of formula: 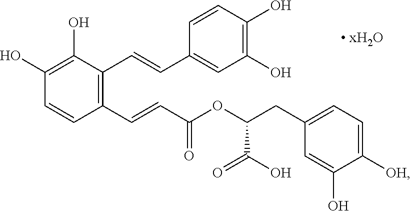 [SMILES: O=C(/C=C/c1ccc(O)c(O)c1/C=C/c1ccc(O)c(O)c1)O[C@H](Cc1ccc(O)c(O)c1)C(=O)O]
and x is 0.5 to 10.

18. The use of any one of claims 10-15, wherein Compound II is Compound IIa: 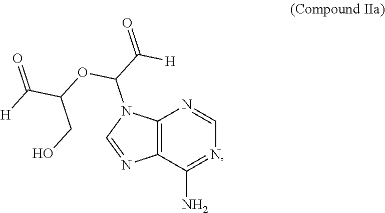 [SMILES: Nc1ncnc2c1ncn2C(C=O)OC(C=O)CO]
or a salt, a hydrate, or a stereoisomer thereof.


SEQUENCE LISTING (3 sequences)

SEQ ID NO: 1 - DNA, 1275 bp, Homo Sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttag

SEQ ID NO: 2 - protein, 424 aa, Homo Sapiens
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 3 - protein, 14 aa, Artificial Sequence
  Synthetic Epitope Tag
GKPIPNPLLGLDST